SEQUENCE LISTING (13 sequences)

SEQ ID NO: 1 - protein, 9 aa, Human immunodeficiency virus
  HIV tat internalisation peptide
RKKRRQRRR

SEQ ID NO: 2 - protein, 16 aa, Drosophila sp.
  Antennapedia cell penetrating peptide
RQIKIWFQNRRMKWKK

SEQ ID NO: 3 - protein, 7 aa, Artificial Sequence
  Nuclear localisation signal
PKKKRKV

SEQ ID NO: 4 - protein, 7 aa, Artificial Sequence
  Scrambled nuclear localisation signal
KKVKPKR

SEQ ID NO: 5 - protein, 7 aa, Artificial Sequence
  Positively charged peptide
GHHHHHG

SEQ ID NO: 6 - protein, 3 aa, Artificial Sequence
  Positively charged peptide (peroxisome targeting)
AKL

SEQ ID NO: 7 - protein, 29 aa, Artificial Sequence
  Positively charged peptide (mitochondrial targeting sequence)
MSVLTPLLLRGLTGSARRLPVPRAKIHSL

SEQ ID NO: 8 - protein, 7 aa, Artificial Sequence
  Positively charged peptide (reverse NLS)
VKRKKKP

SEQ ID NO: 9 - DNA, 25 bp, Artificial Sequence
  Forward primer specific for S100A4 gene
aagttcaagctcaacaagtcagaac

SEQ ID NO: 10 - DNA, 21 bp, Artificial Sequence
  Reverse primer specific for S100A4 gene
catctgtccttttccccaaga

SEQ ID NO: 11 - DNA, 20 bp, Artificial Sequence
  PNA molecule - scrambled PNA sequence
ctttagttatagctctcccc

SEQ ID NO: 12 - DNA, 20 bp, Artificial Sequence
  PNA molecule - PNA sequence directed to tyrosinase 5′UTR
aatgtttgaagaactcaata

SEQ ID NO: 13 - DNA, 20 bp, Artificial Sequence
  PNA molecule - PNA sequence directed to tyrosinase start codon
cagccaggagcattcttcct

(19) United States
(12) Patent Application Publication
(10) Pub. No.: US 2011/0171184 A1
(43) Pub. Date: Jul. 14, 2011

(54) Method for Introducing a Pna Molecule Conjugated to a Positively Charged Peptide into the Cytosol and/or the Nucleus by Photochemical Internalisation (Pci)

(75) Inventors: Eivind Hovig, Oslo (NO); Oystein Fodstad, Oslo (NO); Sigurd Boe, Horten (NO)

(73) Assignee: PCI Biotech AS, Oslo (NO)

(21) Appl. No.: 11/632,024

(22) PCT Filed: Jul. 7, 2005
(86) PCT No.: PCT/GB05/02679

§ 371 Date: Dec. 12, 2007

(30) Foreign Application Priority Data
Jul. 7, 2004 (GB) 0415263.3

(51) Int. Cl. A61K 48/00; C12N 15/87; C12Q 1/68; C12N 15/01; A61P 35/00; C12N 5/00; A61K 38/14; A61K 35/12

(52) U.S. Cl. 424/93.21; 435/455; 435/446; 435/325

(57) ABSTRACT

The present application relates to a method for introducing a PNA molecule into the cytosol, preferably the nucleus of a cell, comprising contacting said cell with a PNA molecule and a photosensitising agent, and irradiating the cell with light of a wavelength effective to activate the photosensitising agent, wherein said PNA molecule is conjugated to a positively charged peptide. Compositions comprising such conjugated PNA molecules, cells made using the method and uses of the method are also described.

The present invention relates to a method for introducing peptide nucleic acid (PNA) molecules conjugated to positively charged peptides into cells, preferably into the cytosol and/or nucleus of cells, using a photosensitising agent and irradiation of the cells with light of a wavelength effective to activate the photosensitising agent, and to the use of this method for assessing or altering gene activity, e.g. antisense or antigene strategies and for downstream applications such as in a high-throughput system for screening the effects of down-regulated gene products.

PNAs are synthetic DNA analogues in which the normal phosphodiester bond found in the DNA backbone is replaced with a 2-aminoethyl-glycine linkage. The nucleotide bases are connected to the uncharged repeating units of the backbone via methyl carbonyl linkers.

As a result of the linkage, PNAs are uncharged. They are also chemically stable and resistant to hydrolytic cleavage, and bind to complementary nucleic acid strands (DNA or RNA) with higher affinity than natural nucleic acids.

Although hybridisation of PNAs to complementary DNA and RNA follows Watson-Crick hydrogen bonding, it is possible to form both parallel and antiparallel duplexes. Furthermore, its hybrid complexes exhibit excellent thermal stability and display unique ionic strength properties. In view of these advantages and the fact that PNAs are resistant to nucleases and proteases, PNAs have been used in in vitro antisense (interfering with translation of mRNA) or antigene (interfering with gene replication or transcription) applications. The PNA-RNA duplexes are not substrates for RNase H and may therefore induce antisense effects based on the steric blocking of either RNA translation or processing. Triplexes result from binding of PNA to DNA which can hinder replication or transcription giving rise to antigene effects. No sign of any general toxicity of PNA has been observed.

Thus, by binding to target nucleic acid molecules, PNAs have significant effects on replication, transcription and translation processes. PNA used in antigene or antisense applications has been shown to hinder the activities of DNA and RNA polymerases, reverse transcriptase, telomerase and the ribosome.

For these effects to be successfully mediated, it is necessary for the PNA molecules to enter the cells and for most applications, the nucleus, which contains some RNA and all DNA except mitochondrial DNA. Cellular and nuclear uptake is however very slow, and does not occur spontaneously. Improving the cellular and nuclear uptake of PNA is therefore a major challenge that has to be overcome before there can be any real prospect of developing it as a therapeutic drug or treatment, or for its widespread application.

One approach to delivering PNA into the cell is to use microinjection (reviewed in Ray and Norden, (2000), FASEB J. 14, 1041-1060). Microinjection is however laborious and time consuming. Furthermore each cell must be individually injected and it is hence most suited to small cell numbers and is not suitable for many in vivo applications. Cell damage is also a problem.

Delivery has also been achieved by electroporation (Shammas et al., (1999), Oncogene 18, 6191-6200), which also has disadvantages, for example it is not suitable for in vivo use.

Membrane disruptive methods such as transient permeabilisation with streptolysin O (Faruqi et al. (1997), P.N.A.S. USA 95, 1398-1403), cell membrane permeabilization by lysolectin (Boffa et al. (1996), J. Biol. Chem. 271, 13228-13233) or detergents like Tween (Norton et al. (1996), Nat. Biotech 14, 625-620) have also been tested. These methods are also not suitable for use in vivo and may cause damage to cells.

Even if it is possible to force PNA molecules into the cell, nuclear uptake may not occur. PNA has been forced to be taken up into cells at high concentrations in vitro, however a very high concentration (of 10 to 20 μM) was required to achieve this (Folini et al. (2003), Cancer Research 63, 3490-3494). It can therefore be seen that improved methods of administering PNA to cells are required.

In vitro cellular delivery of PNA has also been shown to occur when administered with a cationic lipid in the form of a complex. In this particular technique, PNA molecules linked to a functional peptide were hybridised to overlapping oligonucleotides and the complex was mixed with cationic lipid. The cationic lipid-DNA-PNA complex was then internalised, carrying the PNA as a passive cargo (Hamilton et al. (1999), Chem. Biol. 6, 343-351).

PNA lacks the polyanionic charges necessary for condensation and complexation with cationic liposomes through electrostatic interactions. PNA-DNA hybrids, however, possess a distributed negative charge which is contributed by the DNA. Condensed particles can be formed from the interaction of PNA-DNA hybrids with cationic lipids and these lipoplexes are rapidly incorporated into mammalian cells in culture (Borgatti et al. (2003), Oncol. Res. 13(5), 279-287; Borgatti et al. (2002), Biochem. Pharmacol. 64(4), 609-616; Nastruzzi et al. (2000), J. Control Release 68(2), 237-249). PNA may also be transferred into cells by covalent attachment to lipids (Muratovska et al. (2001), Nucleic Acids Res. 29(9), 1852-1863; Ljungstrom et al. (1999), Bioconjug. Chem. 10(6), 965-972; Filipovska et al. (2004), FEBS Lett. 556(1-3), 180-186).

Attempts have also been made to make peptide-PNA constructs that can be taken up by cells in a more efficient manner. Brandén and Smith (2002, Methods in Enzymology 346, 106-124) used a so-called Bioplex system whereby PNA was used to link a functional peptide to DNA to increase the delivery of the DNA. Polyethyleneimine (PEI) may also be added to improve nucleic acid condensation.

This system aimed to provide nucleic acid to the cell and takes advantage of PNA as a means to link the DNA to be delivered to peptides which are designed to improve the delivery of the DNA.

Certain peptides are known to mediate delivery of molecules across the cell membrane. Coupling PNA to such cellular transporter peptides or cell penetrating peptides has also been attempted, to attempt to improve the ability of PNA to enter the cell. Various different transporter peptides have been designed, with the aim of transporting PNA into the cell.

PNA designed as an anti-telomerase agent has been conjugated to the HIV-tat internalisation peptide (SEQ ID NO:1 RKKRRQRRR) and to the Antennapedia cell penetrating peptide (SEQ ID NO:2 RQIKIWFQNRRMKWKK), and shown to have a modest effect as an antisense molecule, reducing telomerase activity. These experiments however only showed a moderate reduction in telomerase activity; Tat-conjugated PNA only reduced telomerase activity to 73% of control level after 48 hours, and Antennapedia conjugated PNA achieved 50% inhibition only at very high concentrations of >30 μM (Folini et al., 2003, supra).

Peptides have also been described that are able to mediate the transport of PNA to the nucleus. Newly synthesised nuclear proteins have been shown to require a particular amino acid sequence in order to reach the nucleus and cross the nuclear membrane. These nuclear localisation signals, when present in proteins not present endogenously in the nucleus may also direct these proteins to the nucleus.

PNA has also been conjugated to a nuclear localisation signal (NLS) (SEQ ID NO:3 PKKKRKV) in attempts to direct the PNA to the cell nucleus. This NLS had been shown to mediate the transfer of SV40 large T antigen across the nuclear membrane. When 10 μM of PNA-NLS was administered to cells, its presence was shown in the nucleus after 24 hours. This effect was shown to be independent of the PNA sequence, but highly dependent on the NLS sequence; a scrambled NLS sequence (SEQ ID NO:4 KKVKPKR) conjugated to PNA showed only minimal amounts of PNA in the nucleus (Cutrona et al., (2000), Nature Biotechnology 18, 300-303). These results were paralleled in functional assays where it was shown that PNA-NLS (wt) (where the PNA is an antigene to myc), inhibited growth of cells, whereas PNA conjugated to a scrambled NLS sequence had effects markedly more similar to the effects of a control PNA.

Brandén et al. (1999, Nature Biotechnology 17, 784-787) similarly showed that while conjugating PNA to peptides could increase nuclear transport of PNA in a NLS sequence dependent manner, no nuclear localisation could be seen following inversion of the NLS sequence.

Further studies have also suggested that for PNA to be successfully transported to the nucleus, it is necessary to conjugate both a cellular membrane transporter peptide and a NLS to the PNA molecule (Braun et al. (2002), J. Mol. Biol. 318, 237-243). The cellular membrane transporter peptide is considered to import the PNA, and the NLS is thought to then further take the PNA to the nucleus. In these experiments, the NLS was shown to be essential for nuclear transport, as constructs containing the cell penetrating peptide with only the peptide sequence lysine-lysine remained in the cytosol.

The interpretation of the above results is complicated by the fact that Richard et al. (J. Biol. Chem., (2003), 278(1), 585-590) have demonstrated that fixation of cells, even under mild fixation conditions, can cause artifacts in such experiments, with nuclear staining being seen in the absence of the PNA in the nucleus under even mildly fixed conditions.

Thus, although it has been shown that under certain conditions, PNA or PNA conjugated to cell penetrating peptides may enter the cell, or as recently shown into endosomes (Richard et al., 2003, supra), in most cases for the biological effect of PNA to be mediated it is necessary for the PNA to translocate to the nucleus.

It can be seen that there remains a need for a reliable and reproducible method of administering PNA molecules such that uptake into the cell is achieved, e.g. the cytosol, preferably the nucleus, without the need to apply high concentrations of PNA.

The inventors have surprisingly shown that PNA molecules that are conjugated to positively charged peptides are endocytosed, and on release from endosomes using the technique of photochemical internalisation (PCI), these molecules are transported to the nucleus.

Thus, in a first aspect the invention provides a method for introducing a PNA molecule into the cytosol, preferably into the nucleus of a cell, comprising contacting said cell with a PNA molecule and a photosensitising agent, and irradiating the cell with light of a wavelength effective to activate the photosensitising agent, wherein said PNA molecule is conjugated to a positively charged peptide.

PCI is a technique which uses a photosensitising agent, in combination with an irradiation step to activate that agent, and achieves internalization of molecules co-administered to the cell. This technique allows molecules that are taken up by the cell into organelles, such as endosomes, to be released from these organelles into the cytoplasm, following irradiation.

The basic method of photochemical internalisation (PCI), is described in WO 96/07432 and WO 00/54802, which are incorporated herein by reference. As set out above, the molecule to be internalised (which for use according to the present invention would be the PNA-peptide conjugate) and a photosensitising agent are brought into contact with a cell. The photosensitising agent and the molecule to be internalised are taken up into a cellular membrane-bound subcompartment within the cell. On exposure of the cell to light of the appropriate wavelength, the photosensitizing agent is activated which directly or indirectly generates toxic species which disrupt the intracellular compartment membranes. This allows the internalized molecule to be released into the cytosol.

These methods use the photochemical effect as a mechanism for introducing otherwise membrane-impermeable molecules into the cytosol of a cell in a manner which does not result in widespread cell destruction or cell death if the methodology is suitably adjusted to avoid excessive toxic species production, e.g. by lowering illumination times or photosensitizer dose.

It is particularly surprising that when the PCI method is used for PNA release into the cell, neither a specific cell penetrating sequence nor a NLS sequence is required for the PNA to enter the cell and for its subsequent translocation to the nucleus. All that is required is that the PNA is coupled to a peptide that has at least a single net positive charge.

Thus, without wishing to be bound by theory it appears that when using PCI, the presence of a positively charged peptide can facilitate the uptake of the PNA molecule into the cell, into cellular compartments such as endosomal compartments, and additionally, following the release or internalisation of the PNA molecule into the cytosol, the charged peptide then additionally mediates the targeting of the PNA molecule to the nucleus. As a consequence of this, only minimal modification of the PNA molecule is required to target it to the desired location and the conjugation of long amino acid sequences or multiple amino acid sequences to the PNA molecule is not necessary.

It is also surprising that only a single peptide is required to perform both of these roles i.e. directing the uptake of the PNA molecule into the cell, and also facilitating the transfer into the nucleus once the PNA-peptide molecule has been released or internalised into the cytosol.

Nuclear localisation signals have been studied in some detail and it has been shown that certain amino acid consensus sequences are required to be present for efficient nuclear targeting. In particular, the importin pathway has been identified as a means by which molecules may be taken to the nucleus. “Classic”, arginine/lysine rich NLSs, such as the SV40T large antigen sequence interact with importin proteins α+β. The complex is translocated through the central channel of the nuclear pore complex and dissociates in the nucleus. The association and dissociation steps are energy dependent mechanisms (reviewed in Cartier et al. (2002), Gene Therapy 9, 157-167). Other pathways for nuclear import are believed to exist, although they have not been so well characterised.

It is surprising therefore that when using the PCI method of the invention not only is a classical NLS sequence not required, but furthermore any sequence with a net positive change of one or more is capable of mediating nuclear localisation. This is demonstrated by the fact that the sequence SEQ ID NO:5 GHHHHHG functioned as well as SEQ ID NO:3 PKKKRKV, and further that the tripeptide with only a single positive change (SEQ ID NO:6 AKL) had the ability to direct PNA first into the endosome, and subsequently into the nucleus (see the examples).

A further, surprising observation is that sequences which were originally identified by virtue of their ability to target proteins to cellular organelles such as peroxisomes and mitochondria, when conjugated to the PNA molecules, are also able to direct the PNA molecules first into the endosome, and subsequently into the nucleus (see the Examples).

The precise role of PCI in the method of the invention is not known, but it is clearly pivotal in the success of the method as without PCI, PNA molecules carrying a positively charged peptide do not enter into the cytosol or nucleus to any significant extent.

The effect also appears to be independent of the overall length of the conjugated peptide, with positively charged peptides of 3 amino acids in length functioning equally as well as those with 29 amino acids. The effect is also independent of the charge to length ratio of the positively charged peptide, and of the particular charged amino acids that are included in the sequence.

As referred to herein, “PNA” refers to a peptide nucleic acid molecule which acts as a DNA analogue and is based on a pseudopeptide skeleton to which nucleotide bases are attached. The PNA may be in a free linear form or may be in a duplexed or self-ligated form, e.g. bis-PNA.

Derivatives of the standard form of PNA are also contemplated, e.g. in which one or more of the pseudopeptide monomers making up the polymer may be modified or derivatized e.g. to provide altered properties, e.g. by using a lysine or other amino acid analogue. Similarly, one or more of the bases used may be modified if desired, e.g. by using non-naturally occurring variants. Thus PNA includes derivatives of the standard form, providing such derivatives retain relevant functional properties, i.e. are capable of forming a sequence-dependent complex with DNA and/or RNA. In other words the PNA derivative is appropriate in terms of charge and structure to allow complementarity to a DNA or RNA sequence.

The PNA molecule may be of any sequence or length. Preferably the PNA molecule is less than 25, e.g. less than 20 bases in length. Preferably the PNA molecule is more than 6 bases in length. For example, molecules of from 6 to 20 bases may be used. A PNA oligomer length of 12 to 17 units is optimal. Sequence length is primarily determined by the required specificity of the method employed. DNA applications that require more than 25 bases can be routinely performed with much shorter PNA probes. Long PNA oligomers, depending on the sequence, tend to aggregate and are difficult to purify and characterize. However, the shorter a sequence is, the more specific it is. Consequently, the impact of mismatch is greater than for a short sequence. PNA oligomers with 20 units have however been used without any aggregation problem.

Such molecules, their chemical properties and methods of synthesis are well known in the art (Ray and Norden, 2000, supra) and they may be prepared by any convenient means.

The PNA molecule may be an antisense PNA molecule or a PNA molecule complementary to a gene (an antigene molecule), which can form a characteristic triplex structure. The PNA molecule may also be a probe, i.e. it may bind to a target nucleic acid sequence and conveniently may carry a label.

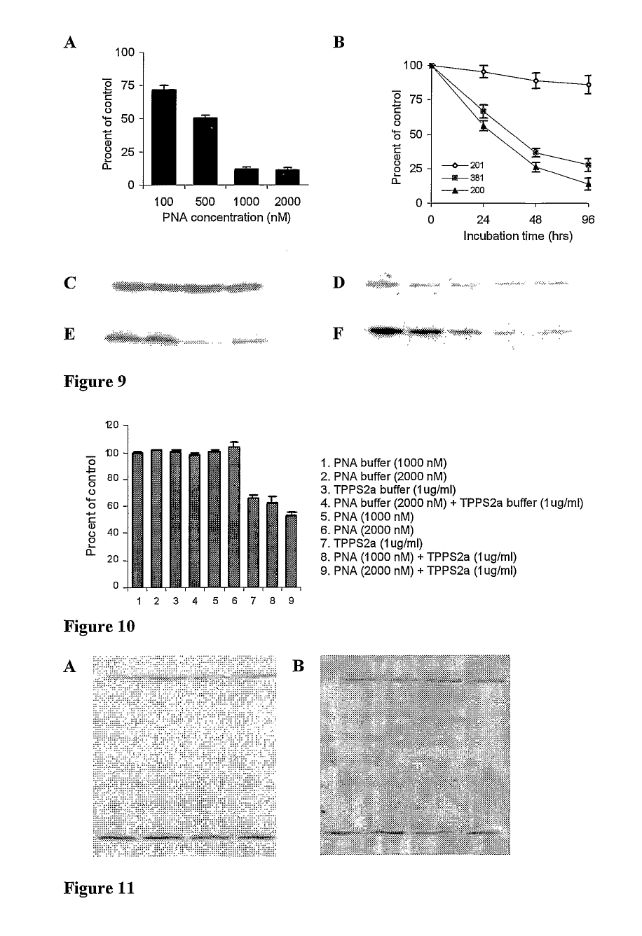

The method of the invention achieves translocation of the PNA-peptide conjugate into the cytosol, preferably into the nucleus. It will be appreciated however that uptake of each and every molecule contacted with the cell is not achievable. Significant and improved uptake relative to background levels in which no PCI is used is however achievable. Preferably methods of the invention allow the uptake of PNA molecules at sufficient levels that their effect on replication, transcription or translation is evident in the expressed products of those cells. The appropriate concentration of PNA-peptide conjugates to be contacted with the cell may be adjusted to achieve this aim, e.g. to achieve a reduction in expression of a target gene of more than 10%, e.g. more than 20, 30, 40 or 50% reduction after incubation with cells for e.g. 24, 48, 72 or 96 hours e.g. 24 to 48 hours (see FIG. 9 for example). The level of reduction of the protein is dependent on the half-life of the protein, i.e. pre-existing protein will be removed in accordance with its half-life. Thus a reduction in expression of more than 10, 20, 30, 40 or 50% is achieved relative to expression at the same time point without PNA so that half-life is taken into account.

The term “cell” is used herein to include all eukaryotic cells (including insect cells and fungal cells). Representative “cells” thus include all types of mammalian and non-mammalian animal cells, plant cells, insect cells, fungal cells and protozoa. Preferably however the cells are mammalian, for example cells from cats, dogs, horses, donkeys, sheep, pigs, goats, cows, mice, rats, rabbits, guinea pigs, but most preferably from humans.

As used herein “contacting” refers to bringing the cells and the photosensitizing agent and/or PNA-peptide conjugate into physical contact with one another under conditions appropriate for internalization into the cells, e.g. preferably at 37° C. in an appropriate nutritional medium.

The photosensitising agent is an agent which is activated on illumination at an appropriate wavelength and intensity to generate an activated species. Conveniently such an agent may be one which localises to intracellular compartments, particularly endosomes or lysosomes. A range of such photosensitising agents are known in the art and are described in the literature, including in WO96/07432, which is incorporated herein by reference. Mention may be made in this respect of di- and tetrasulfonated aluminium phthalocyanine (e.g. AlPcS₂ₐ), sulfonated tetraphenylporphines (TPPSₙ), nile blue, chlorin e₆ derivatives, uroporphyrin I, phylloerythrin, hematoporphyrin and methylene blue which have been shown to locate in endosomes and lysosomes of cells in culture. This is in most cases due to endocytic uptake of the photosensitizer. Thus, the photosensitizing agent is preferably an agent which is taken up into the internal compartments of lysosomes or endosomes. Further appropriate photosensitizers for use in the invention are described in WO03/020309, which is also incorporated herein by reference, namely sulphonated meso-tetraphenyl chlorins, preferably TPCS₂ₐ.

However, other photosensitizing agents which locate to other intracellular compartments for example the endoplasmic reticulum or the Golgi apparatus may also be used. It is also conceivable that mechanisms may be at work in which the effects of the photochemical treatment are on other components of the cell (i.e. components other than membrane-restricted compartments). Thus, for example one possibility may be that the photochemical treatment destroys molecules important for intracellular transport or vesicle fusion. Such molecules may not necessarily be located in membrane-restricted compartments, but the photochemical damage of such molecules may nevertheless lead to photochemical internalisation of the transfer molecules, e.g. by a mechanism in which photochemical effects on such molecules leads to reduced transport of the molecule to be internalized (i.e. the PNA molecule) to degradative vesicles such as lysosomes, so that the molecule to be internalized can escape to the cytosol before being degraded.

Examples of molecules not necessarily located in membrane restricted compartments are several molecules of the microtubular transport system such as dynein and components of dynactin; and for example rab5, rab7, N-ethylmaleimde sensitive factor (NSF), soluble NSF attachment protein (SNAP) and so on.

Classes of suitable photosensitising agents which may be mentioned thus include porphyrins, phthalocyanines, purpurins, chlorins, benzoporphyrins naphthalocyanines, cationic dyes, tetracyclines and lysomotropic weak bases or derivatives thereof (Berg et al., J. Photochemistry and Photobiology, 1997, 65, 403-409). Other suitable photosensitising agents include texaphyrins, pheophorbides, porphycenes, bacteriochlorins, ketochlorins, hematoporphyrin derivatives, and derivatives thereof, endogenous photosensitizers induced by 5-aminolevulinic acid and derivatives thereof, dimers or other conjugates between photosensitizers.

Preferred photosensitising agents include TPPS₄, TPPS₂ₐ, AlPcS₂ₐ, TPCS₂ₐ and other amphiphilic photosensitizers. Other suitable photosensitizing agents include the compound 5-aminolevulinic acid or esters of 5-aminolevulinic acids or pharmaceutically acceptable salts thereof.

“Irradiation” of the cell to activate the photosensitising agent refers to the administration of light directly or indirectly as described hereinafter. Thus cells may be illuminated with a light source for example directly (e.g. on single cells in vitro) or indirectly, e.g. in vivo when the cells are below the surface of the skin or are in the form of a layer of cells not all of which are directly illuminated, i.e. without the screen of other cells.

A “peptide” as defined herein includes any molecule containing any number of amino acids, i.e. one or more amino acids. Preferably however the peptide is a polymer of consecutive amino acids.

Preferably the positively charged peptide is 3 (or 4, 5 or 6) to 30 amino acids in length, more preferably 3 (or 4, 5 or 6) to 25, 3 (or 4, 5 or 6) to 20 or 3 (or 4, 5 or 6) to 15 amino acids in length. In a highly preferred embodiment the peptide is less than 10 amino acids in length, e.g. 3, 4, 5 or 6.

The peptides may be prepared by any convenient means, e.g. direct chemical synthesis or by recombinant means by expressing a nucleic acid molecule of the appropriate sequence in a/cell.

The positively charged molecule is capable of translocating the PNA molecule to which it is conjugated into the cell and then into the cytosol, and preferably also into the nucleus.

As referred to herein, “positively charged” denotes that the overall, or net, charge of the peptide is +1 or higher at physiological pH, i.e. pH 7.2. An amino acid is considered +1 if the predominant species at physiological pH is positively charged when present in the context of the peptide. Each such amino acid in a peptide contributes a further positive charge to calculate the final charge of the peptide. The peptide may contain one or more negatively charged amino acid residues, as well as neutral residues, as long as the net charge of the peptide (calculated by adding together the charge attributed to each amino acid) is positive. The PNA molecule is uncharged and as such does not contribute to the overall charge of the molecule. However it is to be understood that it is the charge of the peptide portion which is important and which is assessed in determining the presence of a positively charged peptide.

The charge of the peptide therefore depends on its amino acid composition. Certain amino acids are charged at normal physiological pH. Positively charged amino acids are lysine (K), arginine (R) and histidine (H) and are considered to be +1 on the above-described scale. Aspartic acid (D) and glutamic acid (E) carry a negative charge at most physiological pHs and are considered −1 on the above scale. Other naturally occurring amino acids are considered to carry no charge. Any number of positively charged or negatively charged amino acids may be present, as long as the overall charge of the peptide is +1 or more.

The amino acids used in peptides for use in the invention need not necessarily be naturally occurring amino acids. One of more of the amino acids in the peptide may be substituted for a non-naturally occurring, e.g. a derivatized amino acid. Such amino acids would similarly be assessed on the basis of their contribution to the charge of the peptide. Thus, as with naturally occurring amino acids, if the predominant species is positive at physiological pH, whether or not that charge is derived from the derivatized portion (e.g. an introduced amine group) or a portion also present in the natural amino acid is irrelevant as long as the overall charge is +1 or more.

The peptide may be present as a portion of a hybrid molecule, e.g. linked to a non-proteinaceous molecule such as an organic polymer which could for example be used as a linking group. The peptide may also be attached to a separate component which may be proteinaceous in nature but which effectively is independent of the peptide, e.g. is uncharged or is in a separate structural configuration. In such cases the peptide would constitute an exposed, preferably peripheral portion and the charge of that portion as the relevant peptide would be assessed.

The positively charged peptide may be conjugated to either the N terminus or the C terminus of the PNA molecule and may be attached with or without a linking group, such as 8-amino-3,6-dioxanoctanoic acid, 2-aminoethoxy-2-ethoxy acetic acid (AEEA) or disulphide linkers. Preferably however the peptide is conjugated directly by covalent binding. Especially preferably no other components are present in the conjugate other than PNA and the peptide.

Previous studies have shown that only classic nuclear localisation signals will transport conjugated molecules to the nucleus. However as mentioned above, it has surprisingly been shown that the nuclear localisation capacity of the peptide is dependent only on charge, and not on sequence, when the method of internalisation is performed using PCI. Peptides with a net change of +5 showed the highest uptake, and this was found to be independent of the sequence contributing that charge. The charge of the peptide is >1, preferably from +1 to +10, e.g. +2 to +8, such as +3 to +6, e.g. +4 or +5.

Preferably peptides for attachment to PNA are rich in K, R and/or H residues. Especially preferably series of consecutive charged residues are used. Preferably other residues used in the peptide are neutral. Thus for example the peptide may have or contain the sequence: Xₙ-(Y)ₘ-Xₒ, in which X are neutral residues and Y is a positively charged residue, which may be the same or different in each position in which they appear, and n, m and o are integers ≧1, e.g. between 1 and 10, and n and o are preferably 1 or 2 and m is preferably from 2 to 5. Especially preferably Y is the same at each position and is K, R or H.

Particularly preferred peptides are SEQ ID NO:7 MSVLTPLLLRGLTGSARRLPVPRAKIHSL, SEQ ID NO:6 AKL and SEQ ID NO:5 GHHHHHG. SEQ ID NO:7 MSVLTPLLLRGLTGSARRLPVPRAKIHSL and SEQ ID NO:6 AKL are mitochondrial and peroxisome targeting sequences, respectively, and yet have proved capable of targeting to the nucleus using the PCI method described herein. This surprising finding illustrates the charge but not sequence-dependency of the peptides which are useful according to the invention.

The positively charged peptide is preferably not a NLS such as SEQ ID NO:3 PKKKRKV or the scrambled NLS SEQ ID NO:4 KKVKPKR or the reverse NLS SEQ ID NO:8 VKRKKKP, or a classic cell penetration peptide such as HIV Tat SEQ ID NO:1 RKKRRQRRR, or the Antennapedia cell penetrating peptide SEQ ID NO:2 RQIKIWFQNRRMKWKK. This may be assessed for example by determining the extent of nuclear transfer or cell penetration without PCI. Peptides capable of significant nuclear transfer or cell penetration under such circumstances would be considered NLS or cell penetration peptides. The peptide is also preferably not polylysine. Additionally, the PNA or the peptide may contain further modifications, such as fluorescent labels on tags.

PNA-peptide conjugates as described above form further aspects of the invention.

As referred to herein “conjugation” refers to the linking together of the peptide and the PNA molecule to form a single entity under physiological conditions.

The PNA and peptide are preferably linked by a covalent bond.

The PNA molecule and the peptide may be synthesised or purified separately and then joined together e.g. using a spacer molecule such as Fmoc-NC603H11-OH (Branden et al., 1999, supra) or they may be chemically synthesised as a single molecule, e.g. by the (Btoc) strategy. In this method, PNA monomers are synthesized into oligomers as long as 20 bases, using protocols for standard peptide synthesis. PNA monomers use fluorenylmethozycarbonyl (Fmoc) protection of the N-terminal monomer amino group, and benzhydryloxycarbonyl (Bhoc) to protect the A, C and G exocyclic amino groups. The Bhoc group coupled with the XAL synthesis handle allows rapid deprotection and cleavage of the PNA oligomer from the resin. Typical coupling yields are >95%. Synthesis is completed by TFMSA cleavage of the oligomer from the resin. The oligomer is purified by reverse phase HPLC (Viirre et al. (2003), J. Org. Chem. 68(4), 1630-1632; Neuner et al. (2002), Bioconjug. Chem. 13(3), 676-678).

Thus, a positively charged peptide appears to be responsible for both taking the PNA into the cell and, once it has been released from the intracellular compartment, for its nuclear uptake.

More than one type of PNA molecule i.e. PNA molecules of different sequences may be administered or introduced simultaneously. Similarly, PNA molecules carrying more than one type of positively charged peptide may be administered or introduced simultaneously.

Optionally, one or other or both of the photosensitising agent and the conjugated PNA molecule to be introduced into cells may be attached to or associated with or conjugated to one or more carrier molecules or targeting molecules which can act to facilitate or increase the uptake of the photosynthesizing agent or the conjugated PNA molecule or can act to target or deliver these entities to a particular cell type, tissue or intracellular compartment. In the case of the conjugated PNA molecule, targeting to the nucleus may already be achieved by the peptide component of the conjugate in accordance with the invention.

Examples of carrier systems include polylysine or other polycations, dextran sulphate, different cationic lipids, liposomes, reconstituted LDL-particles or sterically stabilised liposomes. These carrier systems can generally improve the pharmacokinetics and increase the cellular uptake of the conjugated PNA molecule and/or the photosensitizing agent and may also direct the PNA molecule and/or the photosensitizing agent to intracellular compartments that are especially beneficial for obtaining photochemical internalisation, but they do not generally have the ability to target the PNA molecule and/or the photosensitizing agent to specific cells (e.g. cancer cells) or tissues. However, to achieve such specific or selective targeting the carrier molecule, the PNA molecule and/or the photosensitizer may be associated or conjugated to specific targeting molecules that will promote the specific cellular uptake of the PNA molecule into desired cells or tissues. Such targeting molecules may also direct the PNA molecule to intracellular compartments that are especially beneficial for obtaining photochemical internalization.

Many different targeting molecules can be employed, e.g. as described in Curiel (1999), Ann. New York Acad. Sci. 886, 158-171; Bilbao et al., (1998), in Gene Therapy of Cancer (Walden et al., eds., Plenum Press, New York); Peng and Russell (1999), Curr. Opin. Biotechnol. 10, 454-457; Wickham (2000), Gene Ther. 7, 110-114.

The carrier molecule and/or the targeting molecule may be associated, bound or conjugated to the PNA molecule, to the photosensitizing agent or both, and the same or different carrier or targeting molecules may be used. As mentioned above, more than one carrier and/or targeting molecule may be used simultaneously.

Preferred carrier for use in the present invention include polycations such as polylysine (e.g. poly-L-lysine or poly-D-lysine), polyethyleneimine or dendrimers (e.g. cationic dendrimers such as SuperFect®); cationic lipids such as DOTAP or Lipofectin and peptides.

The method of the invention may be put into practice as described below. In the method of the invention, the molecule to be internalised and a photosensitising compound are applied simultaneously or in sequence to the cells, whereupon the photosensitizing compound and the molecule are endocytosed or in other ways translocated into endosomes, lysosomes or other intracellular membrane restricted compartments.

The PNA-peptide conjugate and the photosensitising compound may be applied to the cells together or sequentially. They may be taken up by the cell into the same or different intracellular compartments (e.g. they may be co-translocated). The PNA-peptide conjugate is then released by exposure of the cells to light of suitable wavelengths to activate the photosensitising compound which in turn leads to the disruption of the intracellular compartment membranes and the subsequent release of the molecule, which may be located in the same compartment as the photosensitizing agent, into the cytosol. Thus, in these methods the final step of exposing the cells to light results in the molecule in question being released from the same intracellular compartment as the photosensitizing agent and becoming present in the cytosol.

More recently, WO 02/44396 (which is incorporated herein by reference) described a method in which the order of the steps could be changed such that for example the photosensitising agent is contacted with the cells and activated by irradiation before the molecule to be internalised and thus delivered to the cell is brought into contact with the cells. This adapted method takes advantage of the fact that it is not necessary for the molecule to be internalised to be present in the same cellular subcompartment as the photosensitising agent.

Thus in a preferred embodiment, said photosensitising agent and said PNA molecule are applied to the cell together or sequentially. As a consequence they may be taken up by the cell into the same intracellular compartment and said irradiation may then be performed.

In an alternative embodiment, said method can be performed by contacting said cell with a photosensitising agent, contacting said cell with the PNA molecule to be introduced and irradiating said cell with light of a wavelength effective to activate the photosensitising agent, wherein said irradiation is performed prior to the cellular uptake of said PNA molecule into an intracellular compartment containing said photosensitising agent, preferably prior to cellular uptake of said molecule into any intracellular compartment.

Said irradiation can be performed after the cellular uptake of the molecule into an intracellular compartment, whether or not said PNA molecule and the photosensitising agent are localised in the same intracellular compartments at the time of light exposure. In one preferred embodiment however irradiation is performed prior to cellular uptake of the molecule to be internalised.

“Internalisation” as used herein, refers to the cytosolic delivery of molecules. In the present case “internalisation” thus includes the step of release of molecules from intracellular/membrane bound compartments into the cytosol of the cells.

As used herein, “cellular uptake” or “translocation” refers to one of the steps of internalisation in which molecules external to the cell membrane are taken into the cell such that they are found interior to the outer lying cell membrane, e.g. by endocytosis or other appropriate uptake mechanisms, for example into or associated with intracellular membrane-restricted compartments, for example the endoplasmic reticulum, Golgi body, lysosomes, endosomes etc.

The step of contacting the cells with a photosensitising agent and with the PNA-peptide conjugate may be carried out in any convenient or desired way. Thus, if the contacting step is to be carried out in vitro the cells may conveniently be maintained in an aqueous medium such as for example appropriate cell culture medium and at the appropriate time point the photosensitising agent and/or PNA-peptide conjugate can simply be added to the medium under appropriate conditions, for example at an appropriate concentration and for an appropriate length of time.

The photosensitizing agent is brought into contact with the cells at an appropriate concentration and for an appropriate length of time which can easily be determined by a skilled person using routine techniques and will depend on such factors as the particular photosensitizing agent used and the target cell type and location. The concentration of the photosensitizing agent must be such that once taken up into the cell, e.g. into, or associated with, one or more of its intracellular compartments and activated by irradiation, one or more cell structures are disrupted e.g. one or more intracellular compartments are lysed or disrupted. For example photosensitising agents used in the Examples herein may be used at a concentration of for example 10 to 50 μg/ml. For in vitro use the range can be much broader, e.g. 0.05-500 μg/ml. For in vivo human treatments the photosensitizing agent may be used in the range 0.05-20 mg/kg body weight when administered systemically or 0.1-20% in a solvent for topical application. In smaller animals the concentration range may be different and can be adjusted accordingly.

The time of incubation of the cells with the photosensitizing agent (i.e. the “contact” time) can vary from a few minutes to several hours, e.g. even up to 48 hours or longer, e.g. 12 to 20 hours. The time of incubation should be such that the photosensitizing agent is taken up by the appropriate cells, e.g. into intracellular compartments in said cells.

The incubation of the cells with the photosensitizing agent may optionally be followed by a period of incubation with photosensitiser free medium before the cells are exposed to light or the PNA molecule is added, e.g. for 10 minutes to 8 hours, especially 1 to 4 hours.

The PNA molecule is brought into contact with the cells at an appropriate concentration and for an appropriate length of time.

Determining the appropriate doses of PNA molecules for use in the methods of the present invention is routine practice for a person skilled in the art. For in vitro applications an exemplary dose of the PNA molecules would be approximately 0.1-500 μg PNA per ml and for in vivo applications approximately 10⁻⁶-1 g PNA per injection in humans. For example, PNA-peptide conjugates may be administered at levels of less than 50 μM, e.g. less than 30 μM, especially preferably less than 10 μM, for example from 0.1 to 1 μM, or 5 to 30 μm, where the concentration indicated reflects the levels in contact with the cell.

As mentioned above, it has been found that the contact may be initiated even several hours after the photosensitising agent has been added and irradiation taken place.

An appropriate concentration can be determined depending on the efficiency of uptake of the PNA molecule in question into the cells in question and the final concentration it is desired to achieve in the cells. Thus “transfection time” or “cellular uptake time” i.e. the time for which the molecules are in contact with the cells can be a few minutes or up to a few hours, for example a transfection time of from 10 minutes until up to 24 hours, for example 30 minutes up to 10 hours or for example 30 minutes until up to 2 hours or 6 hours can be used. Longer incubation times may also be used, e.g. 24 to 96 hours or longer, e.g. 5-10 days.

An increased transfection time usually results in increased uptake of the molecule in question. However, shorter incubation times, for example 30 minutes to 1 hour, seem to result in an improved specificity of the uptake of the molecule. Thus, in selecting a transfection time for any method, an appropriate balance must be struck between obtaining a sufficient uptake of the molecule while maintaining sufficient specificity of the PCI treatment.

In vivo an appropriate method and time of incubation by which the PNA molecule and photosensitizing agents are brought into contact with the target cells will be dependent on factors such as the mode of administration and the type of PNA molecule and photosensitizing agents. For example, if the PNA molecule is injected into a tumour, tissue or organ which is to be treated, the cells near the injection point will come into contact with and hence tend to take up the PNA molecule more rapidly than the cells located at a greater distance from the injection point, which are likely to come into contact with the PNA molecule at a later time point and lower concentration.

In addition, a PNA molecule administered by intravenous injection may take some time to arrive at the target cells and it may thus take longer post-administration e.g. several days, in order for a sufficient or optimal amount of the PNA molecule to accumulate in a target cell or tissue. The same considerations of course apply to the time of administration required for the uptake of the photosensitizing agent into cells. The time of administration required for individual cells in vivo is thus likely to vary depending on these and other parameters.

Nevertheless, although the situation in vivo is more complicated than in vitro, the underlying concept of the present invention is still the same, i.e. the time at which the molecules come into contact with the target cells must be such that before irradiation occurs an appropriate amount of the photosensitizing agent has been taken up by the target cells and either: (i) before or during irradiation the PNA molecule has either been taken up, or will be taken up after sufficient contact with the target cells, into the same or different intracellular compartments or (ii) after irradiation the PNA molecule is in contact with the cells for a period of time sufficient to allow its uptake into the cells. Provided the PNA molecule is taken up into intracellular compartments affected by activation of the photosensitizing agent (e.g. compartments in which the agent is present), the PNA molecule can be taken up before or after irradiation.

The light irradiation step to activate the photosensitising agent may take place according to techniques and procedures well known in the art. For example, the wavelength and intensity of the light may be selected according to the photosensitising agent used. Suitable light sources are well known in the art.

The time for which the cells are exposed to light in the methods of the present invention may vary. The efficiency of the internalisation of the PNA molecule into the cytosol increases with increased exposure to light to a maximum beyond which cell damage and hence cell death increases.

A preferred length of time for the irradiation step depends on factors such as the target, the photosensitizer, the amount of the photosensitizer accumulated in the target cells or tissue and the overlap between the absorption spectrum of the photosensitizer and the emission spectrum of the light source. Generally, the length of time for the irradiation step is in the order of minutes to several hours, e.g. preferably up to 60 minutes e.g. from 0.5 or 1 to 30 minutes, e.g. from 0.5 to 3 minutes or from 1 to 5 minutes or from 1 to 10 minutes e.g. from 3 to 7 minutes, and preferably approximately 3 minutes, e.g. 2.5 to 3.5 minutes.

Appropriate light doses can be selected by a person skilled in the art and again will depend on the photosensitizer and the amount of photosensitizer accumulated in the target cells or tissues. For example, the light doses typically used for photodynamic treatment of cancers with the photosensitizer Photofrin and the protoporphyrin precursor 5-aminolevulinic acid is in the range 50-150 J/cm² at a fluence range of less than 200 mW/cm² in order to avoid hyperthermia. The light doses are usually lower when photosensitizers with higher extinction coefficients in the red area of the visible spectrum are used. However, for treatment of non-cancerous tissues with less photosensitizer accumulated the total amount of light needed may be substantially higher than for treatment of cancers. Furthermore, if cell viability is to be maintained, the generation of excessive levels of toxic species is to be avoided and the relevant parameters may be adjusted accordingly.

The methods of the invention may inevitably give rise to some cell killing by virtue of the photochemical treatment i.e. through the generation of toxic species on activation of the photosensitizing agent. Depending on the proposed use, this cell death may not be of consequence and may indeed be advantageous for some applications (e.g. cancer treatment). Preferably however cell death is avoided. The methods of the invention may be modified such that the fraction or proportion of the surviving cells is regulated by selecting the light dose in relation to the concentration of the photosensitivity agent. Again, such techniques are known in the art.

In applications in which viable cells are desirable, substantially all of the cells, or a significant majority (e.g. at least 50%, more preferably at least 60, 70, 80 or 90% of the cells) are not killed.

Regardless of the amount of cell death induced by the activation of the photosensitiser, for the PNA to have an effect in the cells, it is important that the light dose is regulated such that some of the individual cells wherein the PCI effect is manifested are not killed by the photochemical treatment alone (although they may subsequently be killed by molecules introduced into the cells if those molecules have a cytotoxic effect).

Cytotoxic effects may be achieved by using for example gene therapy in which an antisense PNA molecule is internalized into the nucleus of tumour cells by the method of the invention e.g. to down regulate a gene.

The methods of the invention may be used in vitro or in vivo, for example either for in situ treatment or for ex vivo treatment followed by the administration of the treated cells to the body, for various purposes including (i) inhibition of expression of specific gene products, by binding to mRNA or splicing intermediates; (ii) interfering with transcription of specific genes, by interfering directly with the gene (e.g. inhibiting binding of transcription factors); (iii) as probes for in situ hybridization; (iv) in screening assays; and (v) for achieving site-specific mutagenesis or repair of defective genes inside a target cell.

Thus the present invention provides a method of inhibiting the transcription or expression of a target gene by introducing a PNA molecule into a cell containing said target gene by a method as described hereinbefore, wherein said PNA molecule binds specifically to said target gene or its replication or transcription product. Thus for example said PNA may bind to DNA and/or RNA.

“Specific binding” refers to sequence-dependent binding of PNA to the target molecule be that RNA or DNA. “Target gene” refers to a gene or fragment thereof to which PNA is capable of binding and which is the target of investigation.

The present invention also provides a method of identifying or assessing the level of a target gene or its replication or transcription product, said method comprising introducing a PNA molecule into a cell containing said target gene or its replication or transcription product by a method described hereinbefore, wherein said PNA molecule binds specifically to said target gene or its replication or transcription product, and assessing the levels of bound PNA to determine the existence or level of said target gene or its replication or transcription product. Conveniently for this method the PNA molecule may carry a reporter molecule which may be identified in the assessment, e.g. a radiolabel or means of generating a signal. The assessment may be both qualitative and/or quantitative.

The present invention also provides a method for achieving site-specific mutagenesis or repair of a target gene, preferably a defective gene, in a cell, said method comprising introducing a PNA molecule and an oligonucleotide molecule containing the desired sequence into a cell containing said target gene by a method described hereinbefore, wherein said PNA molecule binds specifically to said target gene to form a PNA clamp. This distortion of the normal nucleic acid forming a triplex occurs at the targeted site and promotes repair or recombination at that particular site. A donor nucleotide, which may be linked to the PNA, or simply administered concomitantly with the PNA, contains the desired nucleotide sequence. The PNA therefore acts as a promoter of repair/recombination.

These methods may be used for exploratory, e.g. diagnostic purposes or to alter the expression profile of cells, e.g. to produce a desired product for isolation or for therapeutic purposes.

The methods of the invention may thus be used for diagnostic purposes where the presence of a particular gene or its replication or transcription product is informative of the presence, stage or prognosis of a disease, condition or disorder. Thus the present invention further provides a method of diagnosing a disease, condition or disorder comprising introducing a PNA molecule into a cell (which may be in vitro, in vivo or ex vivo), by a method as described hereinbefore, wherein said PNA molecule binds specifically to a target gene or its replication or transcription product which is indicative of the presence of said disease, condition or disorder and assessing the level of bound PNA to determine the presence, stage or prognosis of said disease, condition or disorder.

The methods of the invention may also be used in treating any disease which benefits from the down-regulation, repair or mutation of one or more genes. For example, genes that are overexpressed in cancer could be downregulated by administering the appropriate PNA molecule.

PNA inhibiting the expression of a mutant, disease-causing gene could also be administered in combination with a replacement gene (i.e. in gene therapy which involves the therapeutic transfer of genes or the modification of existing genes in a patient's cells), for example in treating cystic fibrosis, cancer, cardiovascular diseases, viral infection and diabetes. Other diseases the treatment of which would benefit from downregulation of one or more genes include leukaemia and pancreatic carcinoma (Cogoi et al. (2003) Nucleosides Nucleotides Nucleic Acids 22(5-8), 1615-1618), amylotrophic lateral sclerosis (AMS) (Turner et al. (2003), Neurochem. 87(3), 752-763), Huntington's disease (Lee et al. (2002) J. Nucl. Med. 43(7), 948-956) and Alzheimer's disease (McMahon et al. (2002) J. Mol. Neurosci. 19(1-2), 71-76).

As described above, PNA may also be used to alter an existing gene, thus may be used to repair a defective gene for example to treat a disease which is causally related to the expression of that defective gene or failure to express the normal form of that gene (Rogers et al. (2002), PNAS U.S.A. 99(26), 16695-16700; Faruqi et al. (1998), PNAS U.S.A. (5(4), 1398-1403).

Thus, a further aspect of the invention provides a composition containing a PNA molecule and optionally separately also a photosensitizing agent as described herein, wherein said PNA molecule is conjugated to a positively charged peptide. In a further aspect the invention provides said composition for use in therapy.

Alternatively described, the present invention provides the use of a PNA molecule as described herein in the preparation of a medicament for treating or preventing a disease, disorder or infection by altering expression of one or more target genes in said patient. Preferably said medicament is for gene therapy, i.e. for treating a disease or disorder which is typified by abnormal gene expression. Said alteration may include down regulation of said expression or upregulation of a modified form of said gene.

According to the different embodiments set out above, the said photosensitizing agent and said PNA molecule is contacted with cells or tissues of a patient simultaneously or sequentially and said cells are irradiated with light of a wavelength effective to activate the photosensitizing agent and irradiation is performed prior to, during or after the cellular uptake of said PNA molecule into an intracellular compartment containing said photosensitizing agent, preferably prior to cellular uptake of said transfer molecule into any intracellular compartment. Thus in an alternative aspect the invention provides a method of treating or preventing a disease, disorder or infection in a patient comprising introducing a PNA molecule into one or more cells in vitro, in vivo or ex vivo according to the methods as described hereinbefore and where necessary (i.e. when transfection is conducted in vitro or ex vivo) administering said cells to said patient, wherein said PNA molecule is conjugated to a positively charged peptide.

As defined herein “treatment” refers to reducing, alleviating or eliminating one or more symptoms of the disease, disorder or infection which is being treated, relative to the symptoms prior to treatment.

“Prevention” refers to delaying or preventing the onset of the symptoms of the disease, disorder or infection.

Compositions of the present invention may also comprise a cell containing a PNA molecule which has been internalised into the cytosol or nucleus of said cell by a method of the invention, wherein said PNA molecule is conjugated to a positively charged peptide. The invention further extends to such compositions for use in therapy, particularly cancer or gene therapy.

Thus, a yet further aspect of the invention provides a cell or a population of cells containing a PNA molecule which has been internalised into the cytosol or nucleus of said cell, which cell is obtainable by a method of the present invention, wherein said PNA molecule is conjugated to a positively charged peptide.

A yet further aspect of the invention provides the use of a such a cell or population of cells for the preparation of a composition or a medicament for use in therapy as described hereinbefore, preferably cancer or gene therapy, wherein said PNA molecule is conjugated to a positively charged peptide.

The invention further provides a method of treatment of a patient comprising administering to said patient cells or compositions of the present invention, i.e. a method comprising the steps of introducing a molecule into a cell as described hereinbefore and administering said cell thus prepared to said patient. Preferably said methods are used to treat cancer or in gene therapy.

In vivo, any mode of administration common or standard in the art may be used, e.g. injection, infusion, topical administration, both to internal and external body surfaces etc. For in vivo use, the invention can be used in relation to any tissue which contains cells to which the photosensitising agent and the PNA molecule are localized, including body fluid locations, as well as solid tissues. All tissues can be treated as long as the photosensitiser is taken up by the target cells, and the light can be properly delivered.

Thus, the compositions of the invention may be formulated in any convenient manner according to techniques and procedures known in the pharmaceutical art, e.g. using one or more pharmaceutically acceptable carrier or excipients. “Pharmaceutically acceptable” as referred to herein refers to ingredients that are compatible with other ingredients of the compositions as well as physiologically acceptable to the recipient. The nature of the composition and carriers or excipient materials, dosages etc. may be selected in routine manner according to choice and the desired route of administration, purpose of treatment etc. Dosages may likewise be determined in routine manner and may depend upon the nature of the molecule, purpose of treatment, age of patient, mode of administration etc. In connection with the photosensitizing agent the potency/ability to disrupt membranes on irradiation, should also be taken into account.

The methods described above may alternatively be used to generate a screening tool for high throughput screening methods, particularly to analyze the effects of silencing a particular gene. PNA directed to one or more specific genes may be generated and used in the method of the invention as described above. The PNA may thus be used to reduce the expression of a gene in a population of cells. The resulting cell population may then be used as a screening tool to identify downstream effects of gene silencing, with standard techniques. Thus for example genes which are also affected by silencing of a target gene may be identified. Optionally such identified genes may be targeted with PNA in further rounds of screening to for example determine the molecules involved in a particular signalling event.

Thus, the invention also relates to a method of screening cells with modified gene expression patterns comprising a) analysing the expression of a target gene or one or more further genes of a cell or a population of cells which have been obtained by the introduction of a PNA molecule according to the method of the invention, wherein said PNA binds specifically to said target gene or its replication or transcription product and modifies the expression of said one or more further genes; and b) comparing the expression of said target and/or one or more further genes to expression of said genes in reference cells, preferably wild type cells.

The expression pattern can be determined using any suitable technique known in the art, e.g. using microarrays carrying probes which bind to mRNA (or cDNA) molecules and may be used to assess the amount of each transcript. Reference cells refer to any cells against which expression is compared. Preferably such cells are control cells to which PNA has not been administered. Especially preferably said cells are wild type cells, e.g. cells that have not been subject to genetic manipulation such as by the use of PNA

Previous attempts to reduce gene expression with normal and chemically modified antisense oligonucleotides have been limited by problems with nuclease degradation of the antisense oligonucleotides, the occurrence of non-specific effects and/or insufficient target affinity. By using the method of the invention to administer PNA, these problems may be overcome.

Thus in a further aspect, the invention provides a method of modifying the gene expression pattern of a cell (e.g. a cell population) to prepare a cell (or cell population) for use as a screening tool (e.g. for high throughput screening), comprising contacting a PNA molecule capable of inhibiting or reducing the expression of a gene, and a photosensitising agent with a cell (e.g. a cell population) and irradiating the cell (e.g. a cell population) with light of a wavelength effective to activate the photosensitising agent, wherein said PNA molecule is conjugated to a positively charged peptide. The invention further extends to such cells and a method of screening such cells wherein specific properties of such cells, e.g. mRNA expression levels of such cells are examined, e.g. in microarrays.

By “modified gene expression pattern” it is meant that as a consequence of the presence of said PNA molecule in the cell nucleus, the transcription or translation of the gene to which it is directed is affected.

As a consequence of this change in expression of the gene, the expression of other genes may be influenced. Thus, by affecting the normal expression of the gene being studied, it is possible to determine the changes in expression pattern of other genes. The identification of these genes, and of the influence that the expression of the gene being studied has on them allows the investigator to draw conclusions about the functions of the gene e.g. their downstream functions. The genes that are affected by the change in normal expression of the gene being studied may be upregulated or downregulated, but the overall change in the pattern of expression gives an indication of the role of the gene in normal cell function and of the consequences of its misregulation.

Using standard techniques well known in the art it is possible to study the effect of the downregulation or elimination of expression of the gene in question. This may for example be done by looking for functional changes in the cells (or cell population), such as changes in cell adhesion, protein secretion or morphological changes. Alternatively, the gene expression profile can be studied directly by analysing mRNA patterns and/or protein expression, again using standard techniques that are well known in the art.

By inhibiting or reducing the expression of a gene, it is to be understood that the expression of the gene in question is reduced, when compared to a cell which has not been subjected to the method i.e. a wild type or normal cell. The change in the level of gene expression may be determined by standard techniques known in the art.

There may be a complete inhibition of expression, such that there is no detectable expression of the gene, i.e. no mRNA or protein is detectable, or there may be a partial inhibition of expression, i.e. a reduction, whereby the amount of gene expression is lower than the wild-type or normal cell. This can be assessed and controlled for by comparing the effect of a PNA with a specific sequence with the effect of a PNA with a scrambled sequence i.e. the same composition of nucleotides, but in a different sequence order. Preferably for this technique to be useful, the reduction in expression is to less than 80% of control levels, e.g. <50%, preferably <20, 10 or 5% of control levels. The cell(s) used will preferably be a cell population, the individual cells of which are genetically identical. The cells may be any cells, as discussed above.

Prior to the development of this new PNA delivery technique it had not been possible to use PNA for such a system. The ability to use PNAs in this system has several advantages. Known techniques for administration of molecules to the cell, such as the use of transfection agents often perturb the cellular assays used in large scale screening systems so that it may be difficult to ascertain which effects are caused by the disruption in gene expression and which are caused by the transfection technique itself. PCI mediated delivery has few such effects, and it is also possible to allow for this by using appropriate controls.

Some of the other substances used for delivery of molecules to the cell may also cause non-specific effects on screening assays. For example short interfering RNA (siRNA), which is used for gene silencing techniques has been reported to affect the expression of interferon genes (Sledz et al. (2003), Natl. Cell Biol. 5(9), 834-839). The stability of PNA is high, and as such the effects that it has on gene expression are prolonged, even after a single administration.

The efficacy of PNA is independent of specific enzyme systems, as its inhibitory action depends on chemical interactions with nucleotide molecules. As such, the degree of inhibition is constant in different cell types. This is not the case for siRNA for example, which is reliant on specific enzymes.

It has been found, surprisingly that the PCI technique does not have the expected problems of generating non-specific effects on gene expression.

The cell or cell population generated according to methods of the invention may be used to make a library which forms a further aspect of the invention.

The invention will now be described in more detail in the following non-limiting Examples with reference to the following drawings in which:

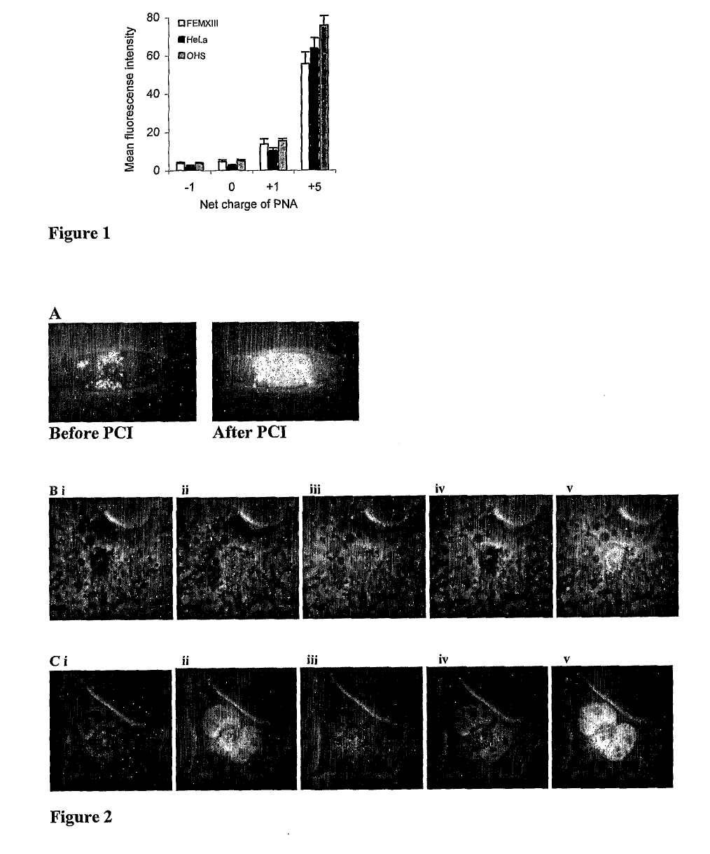

FIG. 1 shows the effect of charge on PNA internalisation using flow cytometry analysis of FITC-PNA uptake into OHS, HeLa and FEMXIII cells. Cells were incubated with 1000 nM of various FITC-PNAs for 24 hrs at 37° C. and analysed by flow cytometry as described in Experimental Protocols. “−1” PNA383, “0” PNA385, “+1” PNA384, “+51” PNA381. Results are shown as mean fluorescence intensity versus net charge of the different PNA molecules. The bars show three individual experiments with 6 parallels each. Error bars show standard deviation of the mean;

FIG. 2 shows relocalization of FITC-PNA-NLS from endocytic vesicles into the nucleus using PCI treatment, using PNA 200 in OHS cells A) Before and after PCI treatment (3 hrs), (B) Before PCI treatment (i) under phase contrast microscopy, (ii) with FITC-PNA staining, (iii) with LysoTracker staining, (iv) with Hoechst staining and (v) showing combined staining, and (C) after PCI treatment with staining as for B;

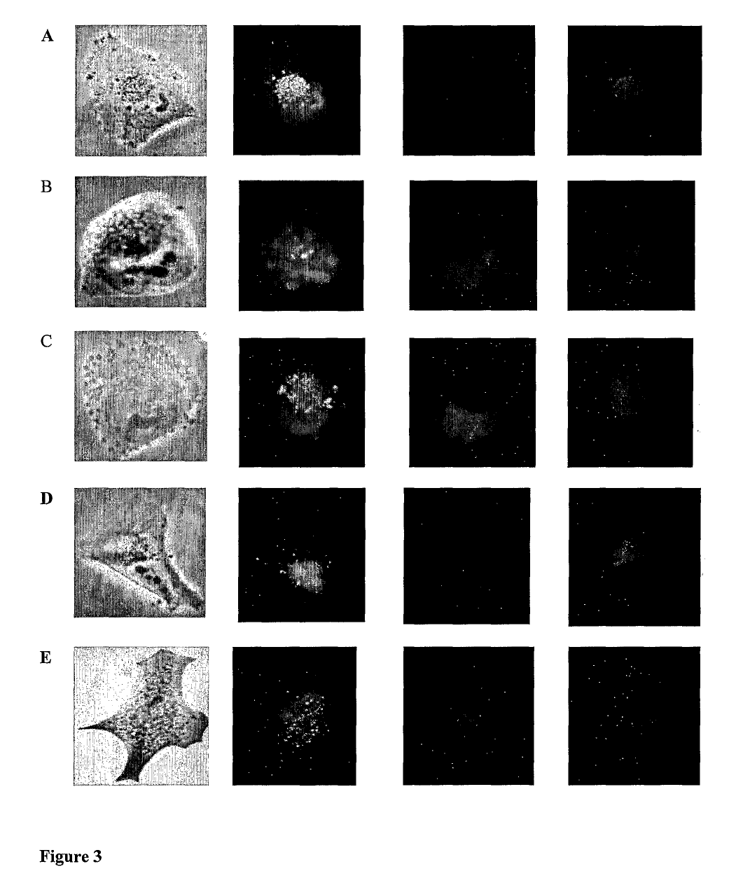

FIG. 3 shows nuclear localisation in different cell types after PCI and using different PNA molecules, using fluorescence microscopy. Cells were incubated with 1000 nM of various FITC-PNAs for 24 hours and analysed by fluorescence microscopy as described in the Experimental protocols. (A) OHS-PNA-NLS (PNA381), (B) OHS-PNA-MITO (PNA382), (C)OHS-PNA-GHHHHHG (PNA457), (D) HeLa-PNA-NLS (PNA381), (E) FEMXIII-PNA-NLS (PNA381), results from left to right, phase contrast image, FITC-PNA, Hoechst, LysoTracker staining;

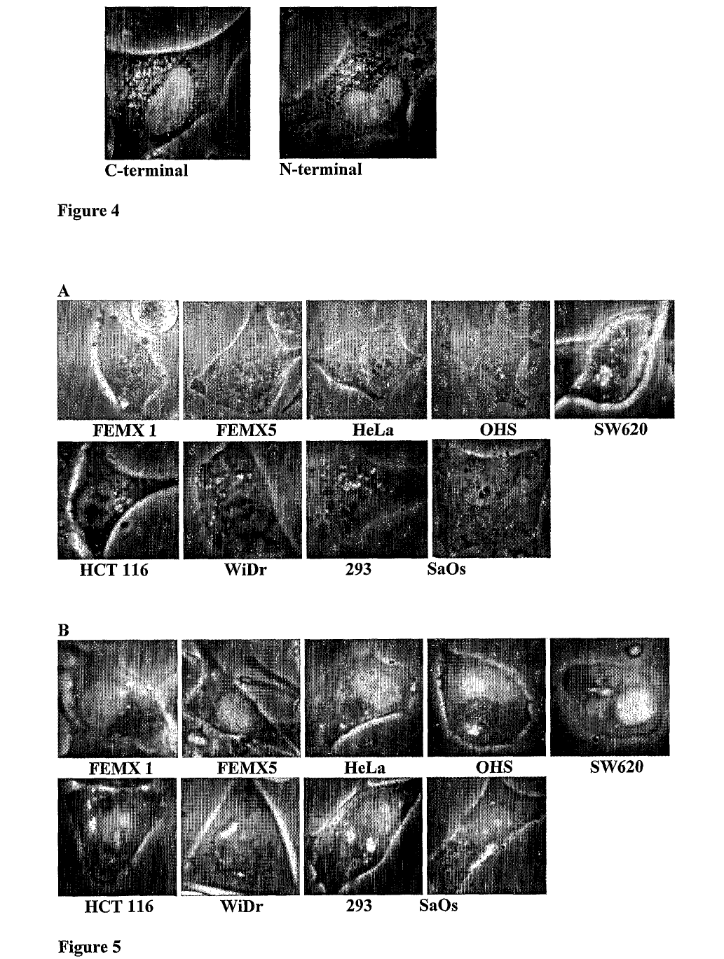

FIG. 4 shows that delivery of PNA to the nucleus is independent of the location of the fluorophore used. OHS cells were incubated with PNA with FITC at the C or N terminus (1000 nM) for 24 hours and analysed by fluorescence microscopy as described in Experimental protocols. Results left and right show FITC linked to either the C or N terminus;

FIG. 5 shows the uptake of PNA 200 into the nucleus of various cells after PCI, (A) before PCI, (B) after PCI, detecting FITC-PNA stain; cells FEMX1, FEMX5, HeLa, OHS, SW620, HCT116, WiDr, 293 and SaOs, respectively;

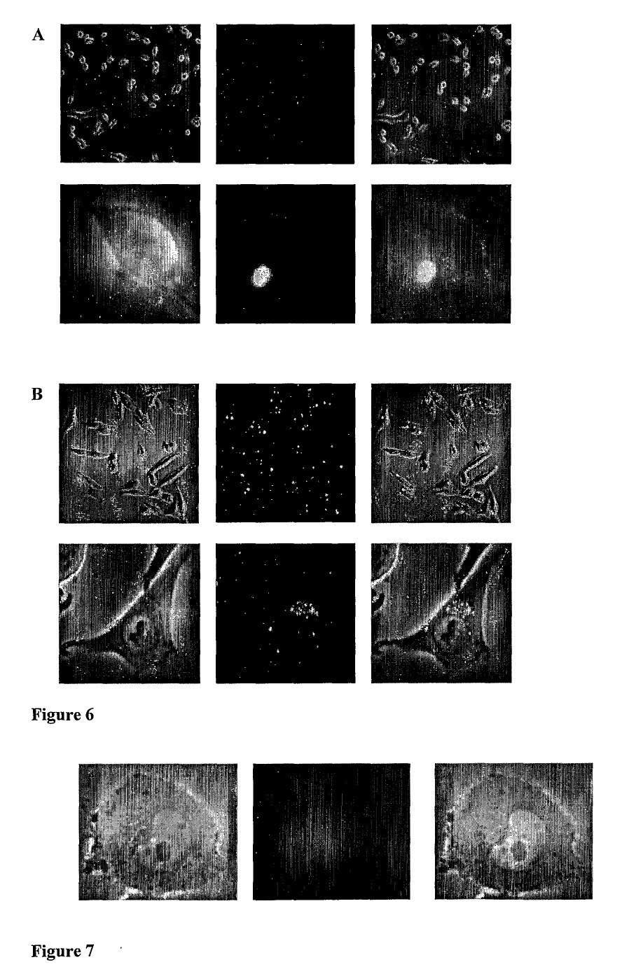

FIG. 6 shows that uptake of PNA is dependent on temperature, OHS cells were exposed to 1000 nM PNA 200 at (A) 4° C. for 5 hours and (B) 37° C. for 5 hours. Results are shown as left to right, phase contrast image, FITC/PNA, combined image; magnification is 10× in the upper pictures and 32× in the lower pictures;

FIG. 7 shows that delivery of PNA to the nucleus is independent of the type of fluorophore used. Cells were incubated with PNA455 (100 mM) conjugated to rhodamine for 24 hours and analysed by fluorescence microscopy as described in the Experimental protocols. Results shown from left to right are the phase contrast image, rhodamine image and combined image;

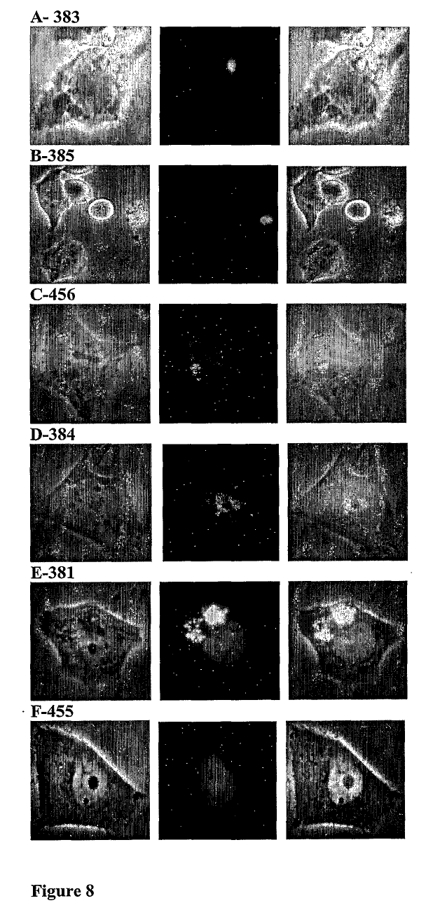

FIG. 8 shows the effect of differently charged PNA molecules on nuclear import after PCI. OHS cells were incubated with PNA (1000 nM) as described in the Experimental protocols (A) 383, (B) 385, (C) 456, (D) 384, (E) 381, (F) 455. Results shown from left to right are phase contrast image, FITC image, combined image;

FIG. 9 shows the inhibition of S100A4 expression in OHS cells by different PNAs (1000 nM) as assessed by Western blotting (A) dose-dependent inhibition with PNA200, (B) time-dependent inhibition by different PNAs. Results are represented as percent of control cells and the bars are the mean from 3 individual experiments. Error bars show standard deviation of the mean. Representative Western blots for the corresponding experiments are shown in C to E; (C) and (D) loading control (α-tubulin), (E) inhibition after 96 hours—from left to right, control, PNA scrambled (PNA201), PNA200, PNA381, (F) dose dependent inhibition after 96 hours with PNA200—from left to right, control, 100 nM, 500 nM, 1000 nM, 2000 nM;

FIG. 10 shows MTS results after PCI treatment of OHS cells. The results indicate that PNAS alone are non-toxic;

FIG. 11 shows Western blot results indicating that no effect on protein levels in OHS cells is seen for PNA targeted to the coding region of S100A4 (PNA452), (A) upper bands—α-tubulin as loading control, lower bands—S100A4, lane 1 control without sensitizer and without light treatment, lane 2 with sensitizer but without light treatment, lane 3 without sensitizer but with light treatment, lane 4 with sensitizer and light treatment, (B) upper bands—α-tubulin as loading control, lower bands—S100A4, lane 1 control, lane 2 PNA scrambled (PNA202), lane 3 (PNA452) 1000 nM, lane 4 (PNA452) 2000 nM;

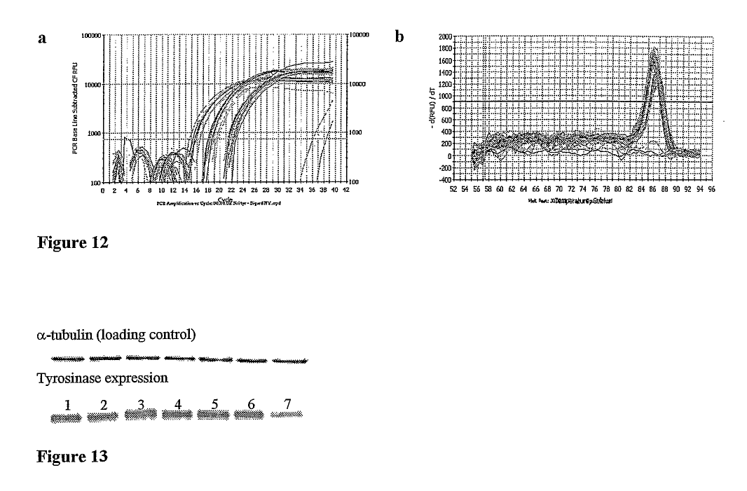

FIG. 12 shows relative expression of S100A4 mRNA in PNA/PCI treated OHS cells vs. control. A) Total RNA was isolated from OHS cells after PNA/PCI treatment with PNA-AUG, PNA-5′UTR and PNA-scrambled. PCI treated OHS cells were selected as a control in addition to PNA scrambled. All samples were reverse transcribed, and dilutions of the cDNA were subjected to real-time PCR analysis using SYBRGreen I as the detection reagent. C_{T}-values obtained from the various samples show little difference in gene expression. B) Melting curve analysis showing only the product of interest.

FIG. 13 shows TYR protein levels after 72 hrs using Western immunoblotting. Lanes were loaded as follows: 1. Control (without PNA), 2. PNA TYR Scrambled (1 mM), 3. PNA TYR Scrambled (10 mM), 4. PNA TYR UTR (1 mM), 5. PNA TYR UTR (10 mM), 6. PNA TYR AUG (1 mM), 7. PNA TYR AUG (10 mM). Alpha tubulin is shown as a loading control.

### EXAMPLES

### Experimental Protocols

### Cell Line and Culture Conditions

The human cell lines HeLa (cervix adenocarcinoma), WiDr (colon carcinoma) and the 293 (embryonic kidney) were obtained from American Type Culture Collection (Manassas, Va., USA). The human OHS (osteosarcoma) and the FEMXIII (melanoma) were established at the Norwegian Radium Hospital (Fodstad et al, (1986), Int. J. Cancer 38(1), 33-40; Fodstad et al., (1988), Cancer Res. 48(15), 4382-8). All cell lines were cultured in RPMI-1640 medium (Bio Whittaker, Verviers, Belgium) except for the 293 cell line which was cultured in DMEM medium (Bio Whittaker, Verviers, Belgium). Both media were used without antibiotics, but supplemented with 10% foetal calf serum (FCS; PAA Laboratories, Linz, Austria) and 2 mM L-glutamine (Bio Whittaker, Verviers, Belgium). The cell lines were grown and incubated at 37° C. in a humidified atmosphere containing 5% CO₂. All cell lines were tested and found negative for Mycoplasma infection.

### PNA Design

PNAs specific for the S100A4 gene including scrambled PNAs were obtained from Oswell DNA Service (Southampton, UK). Modifications were performed at one or both ends (see Table 1). Targets against the 5′UTR (GeneBank accession number NM_{—}002961, 2-15), the AUG start region (63-82) and the coding region in the second exon (98-118) were selected based on previous PNA inhibition studies (Doyle et al., (2001), Biochem. 40, 53-64; Mologni et al., (1999), Biochem. Biophys. Res. Comm. 264, 537-543) and S100A4 gene silencing with ribozymes (Hovig et al., (2001), Antisense Nucleic Acid Drug Dev. April 11(2), 67-75). Sequences were aligned to the human genome database in a BLAST search to eliminate those with significant homology to other genes. Stock solutions (1 mM) were prepared by dissolving PNA in 10% trifluoroacetic acid and heated to 50° C. before use to ensure that PNA was completely dissolved. Prior to use, PNAs were further diluted into working solutions (10 μM) in sterile water and kept at −20° C.

siRNA Design and Annealing

Based on the rules suggested by Elbashir et al. (Elbashir et al, (2001), Genes Dev. 15, 188-200), two targets were selected against the coding region of the S100A4 gene. The first target was against an AA(N)19 sequence (GeneBank accession number NM_{—}002961, 343-361) and the second target was an AA(N19)TT sequence (264-282). In addition, a scrambled control siRNA and a fluorescence labeled siRNA were obtained. All siRNAs were ordered from Eurogentec (Seraing, Belgium). Labeling was performed at both strands, with FITC at the 5′-end of the antisense strand and Rhodamine at the 3′-end sense strand. The GC content of the duplexes was kept within the 40-70% range and all siRNAs were synthesized with dTdT overhang at their 3′ ends for optimal stability of the siRNA duplex. The two target sequences were also aligned to the human genome database in a BLAST search to eliminate those with significant homology to other genes. Dried siRNA oligonucleotides were resuspended to 100 μM in DEPC-treated water and stored at −20° C. Annealing of siRNA was performed by separately aliquotting and diluting each RNA oligo to a concentration of 50 μM. Then 30 μl of each RNA oligo solution and 15 μl of 5× annealing buffer were combined, in a final concentration of 50 mM Tris, pH 7.5, 100 mM NaCl in DEPC-treated water. The solution was then incubated for 2 min in a water bath at 95° C., followed by gradual cooling for 45 min on the workbench. Successful annealing was confirmed by non-denaturing polyacrylamide gel electrophoresis.

siRNA Transfection and PNA Electroporation

OHS cells were cultured as described above and cultivated for 24 hrs in 6 wells plates to 30-60% confluence before transfection. Transfections were performed in serum free OPTI-MEM I medium (Invitrogen Corp, Paisley, UK) with different concentrations of siRNA using Lipofectin Reagent from Life Technologies Inc. (Gaithersburg, Md., USA), Lipofectamine Reagent from Invitrogen (Carlsbad, Calif., USA), (N-(1-(2,3-dioleoxyloxy)propyl)-N,N,N,-trimethylammonium-methyl-sulfate (DOTAP) from Boehringer Mannheim (Mannheim, Germany), FuGene from Roche Diagnostics (Mannheim, Germany), siPORT Lipid Transfection Agent from Ambion (Austin, Tex., USA), and Poly-L-lysine hydrobromide (MW 15.000-30.000) from Sigma (St. Louis, Mo., USA), according to the manufacturer's instructions. In electroporation, the cultured OHS cells were harvested and resuspended into fresh medium. Approximately 4×10⁶ cells were mixed with PNA (1-10 μM) in 300 μl of medium and incubated on ice for 10 min. The cells were electroporated in a 0.4-cm cuvette with a setting of 950 μF/250 V (ECM399, BTX, A Division Of Genetronics, Calif.). Following electroporation, the cells were incubated on ice for 30 min, diluted into T25 flasks and incubated at 37° C. with 5% CO₂ for 24 hrs and analyzed by fluorescence microscopy.

### PCI Technology and Treatment

Sensitizer, Disulfonated Tetraphenylporphine (TPPS₂ₐ) was purchased from Porphyrin Products (Logan, Utah, USA). TPPS₂ₐ was first dissolved in 0.1 M NaOH and thereafter diluted in phosphate-buffered saline (PBS), pH 7.5, to a concentration of 5 mg/ml and a final concentration of 0.002 M NaOH. The photosensitizer was light protected and stored at −20° C. until use. In irradiation, cells treated with TPPS₂ₐ were exposed to blue light with LumiSource prototype (PCI Biotech AS, Oslo, Norway) containing a bank of four fluorescent tubes (Osram 18W/67) with the highest fluence around 420 nm.

Prior to use, cells were cultivated for 24 hrs in 6-wells plates at 37° C. under 5% CO₂. Cells were then incubated with various PNAs and sensitizer TPPS₂ₐ (1 g/ml) for 18 hrs. After uptake, the cells were washed 3 times with fresh medium and incubated in sensitizer-free medium for 4 hrs. Finally, the cells were exposed to blue light for 30 sec and re-incubated for 24, 48 and 96 hrs. Cells were light protected by aluminum foil during the experiment.

### Fluorescence Microscopy

Cells were analyzed by Zeiss inverted microscope, Axiovert 200 equipped with filters for FITC (450-490 nm BP excitation filter, a 510 nm FT beamsplitter, and a 515-565 nm LP emission filter), Rhodamine ( 546/12 nm BP excitation filter, a 580 nm FT beamsplitter, and a 590 nm LP emission filter), and DAPI ( 365/12 nm BP excitation filter, a 395 nm FT beamsplitter, and a 397 nm LP emission filter). Pictures were composed by the use of Carl Zeiss AxioCam HR, Version 5.05.10 and AxioVision 3.1.2.1 software. Organelle-specific markers were used to confirm intracellular PNA localization. Localization of the lysosomes was determined using fluorescence microscope and LysoTracker Red DND-99 (Molecular Probes, Eugene Oreg.). Nuclear localization was determined using Hoechst H33342 (Molecular Probes, Eugene Oreg.).

### Flow Cytometry Analysis

The cells were trypsinized, centrifuged, resuspended in 400 μl of culture medium, and filtered through a 50-μm mesh nylon filter before being analyzed in a FACS-Calibur (Becton Dickinson) flow cytometer. For each sample 10.000 events were collected. FITC labelled PNA was measured through a 510- to 530-nm filter after excitation with an argon laser (15 mW, 488 nm). Dead cells were discriminated from single viable cells by gating on forward scattering vs. side scattering. The data were analyzed with CELLQuest software (Becton Dickinson).

### Western Blotting

Protein lysate's were prepared in 50 mM Tris-HCl (pH 7.5), containing 150 mM NaCl and 0.1% NP-40 with 2 g/ml pepstatin, aprotinin (Sigma Chemical company, St Louis, Mo.) and leupeptin (Roche Diagnostics, Mannheim, Germany). Total protein lysate (30 μg) from each sample was separated by 12% SDS-polyacrylamide gel electrophoresis, and transferred onto Immobilon-P membranes (Millipore, Bedford, Mass.) according to the manufacturer's manual. As a loading and transfer control, the membranes were stained with 0.1% amidoblack. The membranes were subsequently incubated in 20 mM Tris-HCl (pH 7.5), containing 0.5 M NaCl and 0.25% Tween 20 (TBST) with 10% dry milk (blocking solution) before incubation with rabbit polyclonal anti S100A4 (diluted 1:300, DAKO, Glostrup, Denmark) and mouse monoclonal anti alpha-tubulin (diluted 1:250, Amersham Life Science, Buckinghamshire, England) in TBST containing 5% dry milk. After washing, the immunoreactive proteins were visualized using horseradish peroxidase conjugated secondary antibodies (diluted 1:5000 DAKO, Glostrup, Denmark), and the enhanced chemiluminescence system (Amersham Pharmacia Biotech, Buckinghamshire, England). S100A4 protein levels were reported as percentages of control sample and α-tubulin was used as a loading control.

### Measurement of Cell Viability Using MTS Assay

100 μl cells were placed in 96 well plates and left to grow for 24 hours at 37° C. A negative control was also included, containing no cells. 20 μl MTS reagent (tetrazolium salt) was added per 100 μl to each well and incubated for 2 to 4 hours at 37° in the dark. The absorbance was then read at 490 nm.

### Real-Time Reverse Transcriptase PCR

Cells were cultured and treated as described above. After photochemical treatment, cells were incubated with different PNAs for 96 hrs, and then harvested for RNA isolation. Total cellular RNA was isolated with GenElute Mammalian Total RNA Miniprep Kit (Sigma-Aldrich, Steinheim, GER) according to the manufacturer's instructions. For the cDNA synthesis, a primer mix containing 50 pmol oligo-dT, 3 μg total RNA, and dH₂O to 12 μl was prepared for each sample. The mix was denatured for 5 min at 65° C., then quickly cooled on ice and mixed with 18 μl of a reaction mix to a final concentration of 1× First Strand buffer (Invitrogen), 10 mM DTT, 0.3 mM dNTP, 6.5 ng/μl of yeast tRNA and 200 U (6.6 U/μl) of Superscript II enzyme. The cDNA synthesis was performed at 42° C. for 50 min, followed by 15 min inactivation at 72° C.

For the PCR analysis, three ten-fold dilutions were prepared from each cDNA sample, and all reactions were run in triplicate, making a total of 9 PCR tubes per cDNA sample. PCR was performed in a total volume of 25 μl using a final concentration of 3 mM MgCl₂, 200 μM dNTP, 1×PCR buffer, 0.5 U Platinum Taq, 2 μl of cDNA, and 300 nM of primers specific for the S100A4 gene (forward primer 5′-AAGTTCAAGCTCAACAAGTCAGAAC-3′ (SEQ ID NO:9) and reverse primer 5′-CATCTGTCCTTTTCCCCAAGA-3′ (SEQ ID NO:10)). In addition, all reactions were spiked with 1 nM fluorescein as required for the iCycler. Real-time results were obtained using a final dilution of 1:100 000 SYBR Green I (Molecular Probes, Eugene Oreg.) as the detection reagent. Amplification cycles were as follows: 5 min initial denaturation at 95° C. followed by 40 cycles of 15 sec at 95° C./30 sec at 60° C. for product amplification. Real-Time detection of PCR products were achieved using optical 96-well plates and the iCycler iQ Detection System manufactured by Bio-Rad Laboratories, CA. Each sample was set up in triplicates. To detect the amplification of false products or primer dimers (which are equally labeled by SYBRGreen incorporation and would thus affect fluorescence readings), a melting curve, i.e. loss of fluorescence upon denaturation, was included at the end of the PCR amplification protocol. Melting profiles for each sample was compared to those obtained for standard samples.

cDNA Arrays

The microarrays used in this paper were produced in house, using a Micro Grid II robotic printer (Bio Robotics, Cambridge, UK). These 15 k human cDNA arrays were printed on amino silane coated slides (CMT GAPS, Corning Life Sciences, Corning, N.Y.). For details on the array content, we refer to: http://www.med.uio.no/dnr/microarray/index.html.

### RNA Purification and Labelling

Total RNA from cultured cells treated with PCI and different PNAs was isolated as described above. To analyse possible downstream effects as a consequence of S100A4 gene silencing each individual array was hybridised with cDNA from cells treated with active PNA (PNA382 or 453) and scrambled or control PNA (PNA452 (control) or 454 (scrambled)). cDNA was generated from 50 μg total RNA from each of these cell cultures, and differentially labelled with Cy3- or Cy5-dCTP (Amersham Pharmacia Biotech AB) during reverse transcription. The reaction mixture contained anchored oligo-dT 20-mer primers (4 μg), 40 U RNAsin (Promega, Madison, Wis.), 1^{st} strand buffer, 0.01 M DTT, 0.5 mM of dATP, dCTP, dGTP and 0.2 mM dTTP. The mix was incubated in a 65° C. water bath for 5 min. The tubes were then transferred to a 42° C. heating block and 4 μl (4 nmol) of either fluorophore was added to the respective tubes in addition to 400 U Superscript II (Invitrogen, Groningen, The Netherlands). After 60 min, the reaction was inactivated by 5 μl 0.5 M EDTA (pH 8.0). To hydrolyse residual RNA, 10 μl of 1 M NaOH was added, and the tube was incubated at 65° C. for 60 min. 25 μl 1 M Tris-HCl (pH 7.5) was added to neutralize the mixture. Labelled Cy3- and Cy5-cDNA was diluted with 0.5×TE-buffer (pH 7.5) before removing unincorporated dye and concentrating the samples by Microcon YM columns (Ambion, Millipore Corporation, Bedford, Mass.).

### Prehybridization of Slides

The slides were UV-crosslinked at 150 kJ for 60 sec. Immediately prior to use, the slides were prehybridized to inactivate reactive groups on the slide surface and wash away unbound DNA. A small slide holder filled with prehybridization solution was prewarmed at 50° C. for 30 min. The hybridisation solution contained 1% (w/v) Bovine Serum Albumin (BSA) Fraction V (Sigma-Aldrich), 3.5×SSC and 0.1% SDS. The slides were incubated in the prewarmed solution at 50° C. for 25 min immediately following the incubation; the slides were transferred to a clean slide rack and rinsed twice with agitation in ultrapure water at room temperature. To denature the DNA to single stranded form, the slides were agitated in recently boiled water for two minutes, and then quickly immersed in propan-2-ol and agitated for 30 seconds. The slides were dried by centrifugation.

### Hybridisation and Scanning

A hybridisation mixture of 45 μl consisted of 15 μl of each of the labelled probes, 16 μg poly A (Amersham Pharmacia Biotech AB), 4 μg yeast tRNA, 1.25× Denhart's solution, 5 μg BSA, 3.5×SSC (pH 7.5) and 0.3% SDS. The final mix was heated for 2 min at 100° C. and spun down for 10 min at 13 K before it was applied on a microarray slide under the LifterSlip (Erie Scientific Company, Portsmouth, N.H.). The slide was then placed in an ArrayIT hybridisation chamber (Telechem, Sunnyvale, Calif.) and incubated overnight in a water bath at 65° C. Prior to scanning, the coverslip was removed in a solution of 0.5×SSC and 0.1% SDS. The slide was then washed twice in the same solution for 5 min at room temperature, followed by 2 times 5 min in a 0.06×SSC wash solution. The slide was finally dried by centrifugation. Scanning was performed with a ScanARRAY 4000 (Packard Biosciences, Biochip Technologies LLC, Meriden, Conn.) scanner, and data was acquired from the images using GenePix Pro 4.0 software (Axon Instruments Inc., Union City, Calif.). The data were stored, analyzed and processed by use of the BASE (Lao H et al BioArray Software Environment: A Platform for Comprehensive Management and Analysis of Microarray Data Genome Biology 3(8): software0003.1-0003.6 (2002).), and a background-corrected intensity for each spot was calculated by subtracting the median of the pixels in the local background from the mean of the pixels in the spot.

### Example 1

### Cellular Uptake of PNA Molecules

The first set of experiments addressed the question of cellular uptake, i.e. whether PNAs linked to short peptides with different net charge penetrate the cell membrane in different human cancer cell lines or not. To detect the compounds within the cells, PNAs were labeled with FITC at either N- or C-termini. Table 1 details the PNAs used in the study, including their target sequences, chemical modifications and charge.

Cellular uptake was measured by flow cytometry. A PNA linked to a peptide with a net negative charge (PNA383) did not penetrate into OHS cells (FIG. 1, −1 net charge). Since uptake of negatively charged molecules was virtually absent even after 24 hrs, we explored the possibility of transfecting the cells by electroporation. Also in this case, PNA uptake was very poor. In contrast to the negatively charged PNA, neutral PNA with no charge and without any linked peptide (PNA456) and a PNA linked to a peptide with neutral net charge (PNA385) were both internalized at a low level (FIG. 1, 0 net charge for PNA385, PNA456 not shown).

We next investigated the positively charged PNAs. Uptake was clearly observed when PNA was linked to a peptide with +1 net charge (PNA384) (FIG. 1, +1 net charge). However, when we increased the net charge to +5 (PNA381) we observed an almost 5-fold increase in cellular uptake compared to the +1 PNA (FIG. 1, +5 net charge).

The results of the above experiments carried out in OHS, FEMXII and Hela cells are summarized in Table 2.

Staining was used to identify the location of the PNA molecules. Fluorescence of the label attached to the PNA molecule was used to identify the position of the PNA molecule within the cell. Hoechst staining and LysoTracker staining was used to identify the nucleus and lysosomal compartments, respectively. FIG. 2A shows that after PCI, PNA molecules became distributed within the cell. FIGS. 2B and C show that the PNA molecules distribute to the nucleus after PCI (i.e. their distribution is coincident with the Hoechst staining).

In order to explore if cellular uptake was dependent upon conformation of the NLS peptide or just the charge, we linked PNA to the 29 amino-acid long mitochondria import signal with a net charge of +5 (PNA382) (FIG. 3B). As a second control to PNA381 (FIG. 3A), we substituted the original NLS amino acid sequence PKKKRKV (SEQ ID NO:3) with the alternative GHHHHHG (+5) sequence (SEQ ID NO:5, PNA457) (FIG. 3C). The relative levels of cellular uptake for the three different PNA constructs were the same as those revealed by microscopy. In all cases the PNA localized to the nucleus after PCI. This was the case also when conducted in HeLa and FEMXIII cells (FIGS. 3 D and E).

In order to investigate any possible differences in cellular uptake that could be related to the orientation of NLS, we linked the peptide to both the N- (PNA453) and C-termini (PNA381). No difference in uptake levels could be observed (FIG. 4). We also tested the different PNAs and their cellular uptake in various cell lines (HeLa, WiDr, 293, OHS, FEMX5, SW620, HCT116, SaOs), but no significant variation was observed (FIG. 5).

Our data indicate that uptake of chimeric PNAs is strongly dependent upon the net charge of the peptide molecule, and not on the amino acid conformation. We have shown here that uptake of PNA molecules increases as the positive net charge on the conjugated peptide becomes higher.

### Example 2

### PNA Uptake Mechanism and Localization

To assess the uptake process for the modified PNAs, we first explored their uptake under different temperatures. Our results showed no internalization at 4° C. although uptake was seen at 37° C. (FIG. 6). Moreover, our fluorescence microscopy data showed grain like fluorescence spots in a distinct area just in the vicinity of the nuclear envelope (FIG. 6B). Finally, we observed a perfect overlap between the intracellular location of the PNA constructs and a marker for endosomes/lysosomes (LysoTracker Red DND) (data not shown).

Our results show that endocytosis is involved. This may be caused by internalization through coated vesicles.

In our experiments, uptake of our PNAs is blocked at 4° C. Our results are supported by the findings of Kuismanen and Saraste (Kuismanen E et al. (1989) Methods. Cell. Biol. 32, 257-274), which have shown that endocytosis can be blocked at low temperatures. The temperature dependency is further supported by the overlapping localization of PNA and the LysoTracker.

Endocytosis can be divided into several main types: Clathrin-dependent receptor-mediated, clathrin-independent, and phagocytosis. However, further studies have to be carried out to reveal the specific type of endocytosis. A suggestion is that our PNA molecules are taken up by clathrin-independent endocytosis, as there is no evidence of clathrin-dependent receptor-mediated endocytosis. PNA has been linked to different peptide signals with the same charge; the results are the same, indicating that the uptake is not dependent upon a specific receptor. The conclusion is that a positively charged PNA molecule is more likely to have a close association with the cell membrane than a negatively charged one, which in turn will increase endocytic uptake.

### Example 3

### Effect of PCI Treatment

Our microscopy data showed that PNAs with neutral/positive net charge were located in endosomes/lysosomes. Our microscopy data clearly show that PNA constructs are translocated from endosomes/lysosomes to the nucleus after PCI treatment (FIGS. 2 and 3). To confirm the PNA re-localization, we also used a Hoechst nucleus stain, as described above, see FIGS. 2 and 3.

### Example 4

### Nuclear Import of PNA Molecules

For nuclear-based PNA-targeting, major barriers have to be overcome: The most important ones are the cell membrane, the endocytic membranes and the nuclear envelope. After releasing PNA from the endosomes/lysosomes, we wanted to investigate the localization capacity of the NLS peptide and whether the orientation of the NLS peptide was important for nuclear localization. To address these questions, we coupled the NLS peptide to PNAs at both the N- and the C-terminus. Our microscopy data showed that PNAs linked to the NLS peptide at either the N- or C-terminus were translocated to the nucleus (FIG. 4). An increase in fluorescence signal was observed by increasing the time of exposure and the concentration of the FITC/PNA construct. To analyze any possible discrepancies between different types of fluorophores, we exchanged the fluorophore of the PNA from FITC to Rhodamine (Rho). This, however, gave no visible change in localization (FIG. 7. We also linked FITC to either the N- or C-termini of the PNA, again with no change in location or efficiency (FIG. 4).

We next investigated whether the nuclear localization capacity of PKKKRKV (SEQ ID NO:3) simply was due to a change in charge of the PNA, or due to the specific amino acid sequence. In order to control the nuclear localization capacity of the NLS peptide, we tested a PNA with an alternative peptide, having the same net charge (+5), but with substituted amino acids (PNA457, FIG. 3C). Also, we investigated nuclear import of neutral PNAs (PNA456 and 385, FIGS. 8C and B, respectively), and also with PNAs linked to import peptide signals targeted against mitochondria and peroxisomes (PNA382, PNA384, FIGS. 3B and 8D, respectively). Surprisingly, our data demonstrated that all the neutral and positively charged PNAs tested translocated into the nucleus after PCI-treatment.

In summary, our results demonstrated that PNAs with a neutral/positive net charge are not only spontaneously translocated from medium to lysosomes at a high level, but also translocated from the cytosol to the nucleus after photochemical treatment. The efficiency varies between the neutral and the positively charged PNAs, probably as a direct consequence of variations in cellular uptake and not nuclear uptake.

### Example 5

### Inhibition of S100A4 Expression with PNA/PCI

In order to evaluate the capacity of PNA as an inhibitor, we synthesized PNAs directed to three different target sites along the S100A4 gene. We chose a chimeric 14-bp homopurine PNA targeted towards the terminus of the 5′-UTR (PNA381), and two 20-bp mixed-base PNAs towards the start codon (PNA200) and the coding region within the second exon (PNA452). We wanted to investigate whether S100A4 could be down regulated in a dose-dependent manner. We therefore exposed OHS cells to different concentrations (100-2000 nM) of PNA200 for 96 hrs, and checked for viability and for the presence of S100A4 protein by Western blotting (FIGS. 9A and F). Our data clearly demonstrate a dose-dependent inhibition of S100A4 activity, with a decline of signals starting from the concentration of 100 nM using PNA200. Relevant representative controls are shown in FIG. 9D. Furthermore, our results showed that 1000 nM PNA200 caused a maximum inhibition of S100A4 expression. Based on MTS data, which measures mitochondrial integrity as a measurement of cell viability (see Experimental Protocols for details) there is no observable toxicity when using PNA concentrations below 2000 nM. (FIG. 10, lines 5 and 6). Thus, a concentration of 1000 nM PNA concentration was chosen for all subsequent experiments.

To evaluate whether S100A4 protein levels were down regulated in a time-dependent manner, we incubated cells with PNA for 24, 48 and 96 hrs (FIG. 9B). After 24 hrs, the S100A4 protein level was reduced by 45% (PNA200) and 35% (PNA381). Upon longer exposure time (48 hrs), the expression dropped to 25% (PNA200) and 35% (PNA381) compared to control level, respectively.

Finally, S100A4 expression dropped to 10% (PNA200) and 20% (PNA381) compared to control in cells incubated with PNA for 96 hrs after PCI (FIGS. 9B, C and E). Our data indicated that PNAs targeted to both the AUG start site (PNA200) and the terminus of the 5′UTR (PNA381) inhibits S100A4 expression, and that PNA directed against the AUG start site was the most efficient inhibitor. In contrast, we did not detect any inhibition of S100A4 expression by the PNA targeted to the second exon (PNA452), as determined by Western blotting (FIG. 11).

Relative expression of S100A4 mRNA was also examined. Total RNA was isolated from OHS cells after PNA/PCI treatment with PNA-AUG, PNA-5′-UTR and PNA-scrambled. PCI treated OHS cells were selected as a control in addition to PNA scrambled. All samples were reverse transcribed, and dilutions of the cDNA were subject to real-time PCR analysis using SYBRGreen I as the detection reagent. CT-values obtained from the same dilutions showed little difference in gene expression. The results are shown in Table 3.

### Example 6

### Inhibition of S100A4 Expression with siRNA

We compared the ability of PNA to inhibit S100A4 expression with the ability of siRNA to do so. To analyze the siRNA transfection efficiency and distribution, we labelled one of four siRNA with Rhodamine and FITC. We next tested different transfection reagents and concentrations. Our microscopy data displayed no uptake with the use of either FuGene, Lipofectamin, siPORT or Lipofectin (data not shown). However, uptake was demonstrated with both DOTAP and Poly-L-lysine, with Poly-L-lysine as the most effective agent. We therefore used Poly-L-lysine in all subsequent experiments.

In our experiments, siRNA were designed according to Elbashir et al ((2001), Genes Dev. 15, 188-200). In addition to the siRNA designed against the selected target gene, control siRNA was designed by making a scrambled siRNA and a BLAST search was performed against GenBank to eliminate false hybridization. OHS cells were incubated with siRNA for different time periods and concentrations, with subsequent S100A4 protein level measurements performed by Western blots. However, we did not observe any down-regulation in S100A4 expression after 24, 48 and 96 hrs using 20, 50, 100 nM siRNA targeted to two regions in the S100A4 gene (data not shown).

PNA may work by arresting transcriptional processes by virtue of their ability to form stable triplex structures, a strand-invaded or a strand displacement complex with DNA. Such complexes can create structural hindrances to block the stable function of RNA polymerase, and may thus be capable of working as antigene agents. At the level of translation, the PNA antisense effect is based on the steric blocking of either RNA processing, transport into cytoplasm, or translation. The inability of PNAs to activate RNase H eliminates the likelihood of unintended degradation of non-target mRNAs. Additionally, the lack of a negatively charged backbone prevents PNAs binding to the many proteins inside and outside of cells that normally act to bind negatively charged macromolecules. The inhibitory effect of PNA381 is in agreement with Doyle et al. (2001, supra), who demonstrated that PNAs targeted to the terminus of the 5′-UTR were efficient inhibitors in the luciferase mRNA. Furthermore, translation experiments performed in cell-free extracts showed that PNA blocked translation in a dose-dependent manner when targeted close to the AUG start codon of RNA (Knudsen & Nielsen (1996), Nucleic Acids Res. 24, 494-500). No effect was seen when the PNA was targeted towards sequences in the coding region. These results support our results with PNA200 and PNA452, targeted towards the AUG start site and the second exon of S100A4, respectively (FIGS. 9 and 11). The patterns of S100A4 expression in cells exposed to scrambled PNA201/202 and control cells without PNA, but with sensitizer are virtually identical to untreated cells. Additionally, we tested S100A4 expression in OHS cells with or without sensitizer. Again, no differences in protein levels could be observed (FIG. 11A).

### Example 7

### Real-Time Reverse Transcriptase PCR Analysis

To investigate the underlying mechanism of gene silencing, we measured the relative S100A4 mRNA levels by Real Time RT-PCR before and after PNA/PCI treatment. The purpose was to investigate whether our PNA molecules executed their effect at the level of transcription, or at some other level of the protein synthesis process. As can be seen from the amplification figure, there were no distinct differences between the C_{T} values obtained from the PNA/PCI treated samples and the untreated control 96 hrs post treatment (FIG. 12). The scrambled PNA201 was used as internal PNA control for PNA200 and PNA381, respectively.

Previously, Demidov et al. ((1995) Proc. Natl. Acad. Sci. U.S.A., 92, 2637-2641) studied the kinetics and mechanism of PNA binding to duplex DNA. Results showed that formation of a triplex invasion complex is dependent upon homopyrimidine PNAs binding to a homopurine DNA target. A second complex, called the duplex invasion complex, can also be formed, but with homopurine PNAS. The conventional triplex seems to be formed only with cytosine-rich homopyrimidine PNAs. These results imply that the only PNA molecule that we have used which is able to target duplex DNA is the homopurine PNA381 targeted to the terminus of the 5′-UTR. However, even though our homopurine PNA (PNA381) is designed to bind to both DNA and RNA, Real-Time RT-PCR data indicate that it is operating at the level of translation. The other PNAs with mixed-base composition are according to the theory not capable of forming a triplex or duplex invasion complex that is necessary for arresting transcriptional processes. This is in agreement with our Real-Time RT-PCR results and supports the theory that the mixed-base PNA (PNA200) is unable to arrest transcriptional processes.

### Example 8

### Microarray Analysis

In order to examine possible effects of S100A4 inhibition on gene transcription in cDNA microarray experiments, we compared PNA-treated cells with cells undergoing the exact same treatment at the same time, but with a scrambled PNA. PNA molecules directed to two target sequences on the gene were examined. All experiments were performed in duplicate. Only a small number of gene's consistently displayed a relative change in expression of more than two-fold. Using hierarchical clustering, a cluster was identified that displayed a pattern of consistent upregulation when treated with the PNA that caused the largest reduction in S100A4 levels, and a smaller upregulation with the less effective PNA. This cluster contained nine named genes (GAS2, UBE4B, FREQ, SHC1, PON3, CTSD, WNT3A, SCD and RAB6A). These are genes involved in processes including stress response, apoptosis and calcium binding. The levels of transcript downregulation were verified using real-time PCR. To demonstrate that the observed changes were the result of the sequence specific effect of the PNA on the target gene, real-time PCR performed on each step of the process separately, supported this conclusion (data not shown).

As a first demonstration of the ability to utilize PNA/PCI/LS for systematic gene silencing, we examined the global mRNA expression level changes using cDNA microarrays. Because the effects on gene expression of PNA additives and/or photochemical treatment are presently not known, we performed microarray experiments using cells treated with a scrambled PNA as the reference channel. This was done in order to minimize the potential confounding influence of the treatment regimen. As minor variation in handling, exposure times, etc. may still occur, we performed real-time PCR on each step of the treatment process to rule out that the observed expression changes were a consequence of the treatment rather than an S100A4 specific effect. Particularly, PCI could lead to transcriptional changes related to processes including apoptosis (Ferreira S. D. et al, 2004, Lasers Med Sci 18(4): 207-12). Thus, as a general tool, PNA/PCI/LS would be thought to be less well suited for monitoring of gene silencing of genes related to such processes. However, the strategy has a number of appealing aspects, in ease of target sequence design, PNA stability, high throughput synthesis and administration, and timed delivery makes this system a good choice for systematic in vitro silencing of cell lines. siRNA gene silencing has also been demonstrated to be a highly viable strategy, but with some problems related to target sequence design and stability (Amarzguioui M et al, 2004: Biochem. Biophys. Res. Commun. 316(4): 1050-8).

The ability of S100A4 to modulate gene expression is generally unknown, but as this is a protein suggested to be involved in cytoskeleton remodelling, and as such a cell structure protein rather than a regulator of transcription, relatively minor effects would be expected. Accordingly, the transcript level changes seen were relatively minor in terms of both amplitude and in terms of the number of genes affected. Among the genes for which a consistent change could be observed, frequenin is especially interesting, being a calcium binding protein with four EF hands (polyclonal antibody available from www.abcam.com).

### Example 9

### Gene Silencing of the Tyrosinase Gene (TYR) in the Melanoma (FEMX V) Cell Line Using PNA/PCI

We have designed PNAs against different genes involved in melanin biosynthesis; including tyrosinase (TYR), tyrosinase related protein 1 (TRP-1), and microphthalmia transcription factor (MITF). Our goal is to silence all three genes by using the PNA/PCI method. TYR, TRP-1 and MITF are linked to each other, which makes them interesting as a model system. Since these genes are connected to each other it will be very interesting to investigate possible effects at TYR/TRP-1 protein levels when we knock down MITF.

### Cell Line and Culture Conditions

The FEMX V (melanoma) cells were established at the Norwegian Radium Hospital. Cells were cultured in RPMI-1640 medium (Bio Whittaker, Verviers, Belgium). Media were used without antibiotics, but supplemented with 10% fetal calf serum (FCS; PAA Laboratories, Linz, Austria) and 2 mM L-glutamine (Bio Whittaker, Verviers, Belgium). Cells were grown and incubated at 37° C. in a humidified atmosphere containing 5% CO₂. Cells were tested and found negative for Mycoplasma infection.

### PNA Design

PNAs specific for the tyrosinase (TYR) gene including scrambled PNAs were obtained from Oswell DNA Service (Southampton, UK). Modifications were performed at both ends (with FAM and NLS sequence). Targets against the AUG start codon were selected based on previous PNA inhibition studies. Sequences were aligned to the human genome database in a BLAST search to eliminate those with significant homology to other genes.

The following PNA sequences were used:

| | |
|---|---|
| CTTTAGTTATAGCTCTCCCC | (TYRSCR- SEQ ID NO: 11) |
| AATGTTTGAAGAACTCAATA | (TYR5UTR- SEQ ID NO: 12) |
| CAGCCAGGAGCATTCTTCCT | (TYRATG- SEQ ID NO: 13) |

Each PNA molecule was labeled with FAM at the N-terminal (5′-end) and a NLS peptide at C-terminal (3′ end), i.e. FAM-L-L-PNA-L-L-PKKKRKV, where L is a linker (2-aminoethoxy-2-ethoxy acetic acid (AEEA)). Stock solutions (1 mM) were prepared by dissolving PNA in 0.1% trifluoroacetic acid and heated to 5° C. before use to ensure that PNA was completely dissolved. Prior to use, PNAs were further diluted into working solutions (100 mM) in sterile water and kept at −20° C.

The PNA molecules were administered to FEMX V cells using the PCI method as described in Example 6, and protein levels were determined by Western Blotting.

The results as shown in FIG. 13 show that TYR can be silenced by the PNA/PCI method, however, further optimization will lead to a more powerful gene silencing effect. In particular, lane number 7 shows down-regulation of TYR protein after incubation with 10 mM PNA targeted against the start codon region.

1. A method for introducing a PNA molecule into a cell, comprising contacting said cell with a PNA molecule and a photosensitising agent, and irradiating the cell with light of a wavelength effective to activate the photosensitising agent, wherein said PNA molecule is conjugated to a positively charged peptide.

2. The method of claim 1, for introducing the PNA molecule into the nucleus of the cell.

3. The method of claim 1 wherein said PNA molecule is less than 25 bases in length.

4. The method of any one of claim 1 wherein said PNA molecule is an antisense molecule, is complementary to a gene or is a probe.

5. The method of claim 1 wherein the introduction of said PNA molecule is at a concentration to cause a reduction in expression of a target gene of more than 10% after incubation with cells for 24 hours.

6. The method of claim 1 wherein said cell is a eukaryotic cell, preferably a mammalian cell.

7. The method of claim 1 wherein said photosensitising agent is selected from TPPS4, TPPS23, AIPcS2a, TPCS235-aminolevulinic acid and esters of 5-aminolevulinic acid.

8. The method of claim 1 wherein said positively charged peptide is a polymer of consecutive amino acids.

9. The method of claim 1 wherein said positively charged peptide is 3 to 30 amino acids in length.

10. The method of claim 1 wherein said positively charged peptide is conjugated directly to the PNA molecule by covalent linking.

11. The method of claim 1 wherein said positively charged peptide has a charge of from +1 to +10, preferably from +3 to +6.

12. The method of claim 1 wherein said positively charged peptide comprises the sequence Xn-(Y)1n-X0, wherein X is a neutral residue and Y is a positively charged residue which may be the same or different in each position in which it appears, and n, m and o are integers>1.

13. The method of claim 12 wherein Y is the same at each position and is K, R or H.

14. The method of claim 1 wherein the positively charged peptide is SEQ ID NO: 7 MSVLTPLLLRGLTGSARRLPVPRAKIHSL, SEQ ID NO: 6 AKL or SEQ ID NO: 5 GHHHHHG.

15. The method of claim 1 wherein more than one type of PNA molecule is introduced simultaneously, wherein each type has a different sequence.

16. The method of claim 1 wherein one or both of the photosensitising agent and the PNA molecule is attached to or associated with or conjugated to one or more carrier molecules or targetting molecules.

17. The method of claim 16 wherein said carrier molecule or targetting molecule is a polycation, a cationic lipid, lipofectin or a peptide.

18. The method of claim 1 wherein said photosensitising agent and said PNA molecule are applied to the cell together or sequentially.

19. The method of claim 1 wherein said method is performed by contacting said cell with a photosensitising agent, contacting said cell with the PNA molecule to be introduced and irradiating said cell with light of a wavelength effective to activate the photosensitising agent, wherein said irradiation is performed prior to the cellular uptake of said PNA molecule into an intracellular compartment containing said photosensitising agent, preferably prior to cellular uptake of said molecule into any intracellular compartment.

20. The method of claim 1 wherein irradiation is performed for up to 60 minutes.

21. The method of claim 1 performed in vitro or ex vivo.

22. The method of claim 1, for inhibiting the transcription or expression of a target gene in the cell, wherein said PNA molecule binds specifically to said target gene or its replication or transcription product.

23. The method of claim 1, for identifying or assessing the level of a target gene or its replication or transcription product, wherein said PNA molecule binds specifically to said target gene or its replication or transcription product, further comprising assessing the levels of bound PNA to determine the existence or level of said target gene or its replication or transcription product.

24. The method of claim 1, for achieving site-specific mutagenesis or repair of a target gene, including a defective gene, in the cell wherein said PNA molecule binds specifically to said target gene to form a PNA clamp.

25. The method of claim 1, for diagnosing a disease, condition or disorder, wherein said PNA molecule binds specifically to a target gene or its replication or transcription product which is indicative of the presence of said disease, condition or disorder, further comprising assessing the level of bound PNA to determine the presence, stage or prognosis of said disease, condition or disorder.

26. The method of claim 1, for treating a disease which benefits from the down-regulation, repair or mutation of one or more genes, wherein said PNA specifically binds to one or more of said genes, wherein said disease is cancer, cystic fibrosis, cardiovascular disease, viral infection, diabetes, amylotrophic lateral sclerosis, Huntington's disease or Alzheimer's disease.

27. (canceled)

28. A composition comprising a PNA molecule and a photosensitising agent, wherein said PNA molecule is conjugated to a positively charged peptide.

29. The composition of claim 28, further comprising a cell or a population of cells.

30-33. (canceled)

34. The method of claim 1, for treating or preventing a disease, disorder or infection in a patient, wherein said contacting is performed in vivo.

35. The method of claim 1, for treating or preventing a disease, disorder or infection in a patient, wherein said contacting is performed in vitro or ex vivo, further comprising the step of administering the cell or a population of the cells to said patient.

36. The method of claim 34, wherein said method is used to treat cancer or is used in gene therapy.

37. (canceled)

38. A method of screening cells with modified gene expression pattern comprising a) contacting one or more cells with a PNA molecule and a photosensitising agent, and irradiating the cell with light of a wavelength effective to activate the photosensitising agent, wherein said PNA molecule is conjugated to a positively charged peptide; b) analysing the expression of a target gene or one or more further genes of the cell, wherein said PNA binds specifically to said target gene or its replication or transcription product and modifies the expression of said one or more further genes; and c) comparing the expression of said target and/or one or more further genes to expression of said genes in one or more reference cells, including wild type cells.

39. The method of claim 38 wherein expression of said target gene is reduced to less than 80% of control (wild type) levels.

40. The method of claim 1, for introducing the PNA into the cytoplasm of the cell.
